(19) United States
(12) Patent Application Publication
(10) Pub. No.: US 2026/0113825 A1
(43) Pub. Date: Apr. 23, 2026

(54) CONTROL METHOD FOR PULSED LIGHT DEVICE AND RELATED DEVICE

(71) Applicant: Hangzhou Ulike Technology Co., Ltd., Hangzhou (CN)

(72) Inventor: DI ZENG, Hangzhou (CN)

(21) Appl. No.: 19/153,122

(22) PCT Filed: Nov. 8, 2024
(86) PCT No.: PCT/CN2024/131057

§ 371 Date: Aug. 1, 2025

(30) Foreign Application Priority Data
Apr. 3, 2024 (CN) 202410406025.5

(51) Int. Cl. H05B 47/16; A61B 17/00; A61N 5/06; H02J 7/34

(52) U.S. Cl. CPC H05B 47/16; A61N 5/0617; H02J 7/345; A61B 2017/00194; A61B 2017/00752; H02J 2207/50

(57) ABSTRACT

A pulsed light device and a related device are provided. The pulsed light device includes a charging module, a capacitor, and a light source. The capacitor is connected between the charging module and the light source, the charging module is configured to charge the capacitor, and the capacitor is configured to supply power to the light source to allow the light source to flash. The control method includes: controlling the light source to flash N times in one flash window, N being an integer greater than or equal to 3; controlling the capacitor to discharge to the light source during each flash of the light source; and controlling the charging module to charge the capacitor during a time interval between two adjacent flashes.

### TECHNICAL FIELD

The present disclosure relates to the technical field of pulsed light, and in particular to a control method for a pulsed light device and a related device.

### BACKGROUND

In the field of beauty and personal care, the pulsed light device has been widely used. To generate a pulsed light of sufficient intensity in a short period of time, a capacitor is commonly used to power the pulsed light device. The capacitor converts all the stored energy into a strong pulse of light in a single discharge process. However, the high energy produced by a one-time discharge not only increases the user's discomfort, such as pain and other adverse reactions, but also affects the lifespan of sensitive components in the device, reducing its durability of the device.

### SUMMARY

Embodiments of the present disclosure provide a control method for a pulsed light device and a related device, which can control the light source to flash multiple times, so as to disperse the output of energy, thereby improving the user experience and prolonging the service life of the device.

According to a first aspect, an embodiment of the present disclosure provides a control method for a pulsed light device. The pulsed light device includes a charging module, a capacitor, and a light source. The capacitor is connected between the charging module and the light source, the charging module is configured to charge the capacitor, and the capacitor is configured to supply power to the light source to allow the light source to flash. The control method includes: controlling the light source to flash N times in one flash window, N being an integer greater than or equal to 3; controlling the capacitor to discharge to the light source during each flash of the light source; and controlling the charging module to charge the capacitor during a time interval between two adjacent flashes.

In some embodiments, the controlling the light source to flash N times in one flash window includes: controlling the light source to flash M times, M being an integer greater than 1; controlling the charging module to charge the capacitor for a preset duration; and controlling the light source to flash K times, K being an integer greater than 1, and N being greater than or equal to the sum of M and K; wherein a time interval between adjacent flashes in the M flashes and a time interval between adjacent flashes in the K flashes are both less than or equal to the preset duration.

In some embodiments, the preset duration is greater than or equal to 0.4 seconds and less than or equal to 0.95 seconds.

In some embodiments, the controlling the light source to flash N times in one flash window includes: controlling the light source to flash M times, M being an integer greater than or equal to 1; controlling the charging module to charge the capacitor for a preset duration, the preset duration being greater than or equal to 0.4 seconds and less than or equal to 0.95 seconds; and controlling the light source to flash K times, K being an integer greater than or equal to 1, and N being greater than or equal to the sum of M and K.

In some embodiments, in a configuration that M is greater than 1, the time interval between adjacent flashes in the M flashes is greater than or equal to 0.06 seconds and less than or equal to 0.4 seconds; and/or, the preset duration is greater than or equal to 0.5 seconds and less than or equal to 0.7 seconds; and/or, in a configuration that K is greater than 1, the time interval between adjacent flashes in the K flashes is greater than or equal to 0.06 seconds and less than or equal to 0.4 seconds.

In some embodiments, in the configuration that M is greater than 1, the time interval between adjacent flashes in the M flashes is greater than or equal to 0.1 seconds and less than or equal to 0.3 seconds; and/or, in the configuration that K is greater than 1, the time interval between adjacent flashes in the K flashes is greater than or equal to 0.1seconds and less than or equal to 0.3 seconds.

In some embodiments, a flash duration of each flash of the light source is greater than or equal to 0.3 milliseconds and less than or equal to 10 milliseconds; and/or, the pulsed light device is a hair removal device or a skin rejuvenation device.

In some embodiments, the flash duration of each flash of the light source is greater than or equal to 0.5 milliseconds and less than or equal to 4 milliseconds.

In some embodiments, in a configuration that M is equal to 2, a flash duration of a first flash in the M flashes is equal to or less than a flash duration of a second flash in the M flashes; and/or, in a configuration that K is equal to 2, a flash duration of a first flash in the K flashes is equal to or less than a flash duration of a second flash in the K flashes.

In some embodiments, in a configuration that K is equal to 2, a flash duration of a first flash in the K flashes is less than a flash duration of a second flash in the K flashes, the flash duration of the first flash in the K flashes is greater than or equal to 0.3 milliseconds and less than or equal to 0.7 milliseconds, and the flash duration of the second flash in the K flashes is greater than or equal to 1.2 milliseconds and less than or equal to 3 milliseconds.

In some embodiments, the pulsed light device further includes a level setting module provided with a plurality of levels; the control method further includes: determining a target level in response to a user selecting the target level among the plurality of levels by the level setting module; and the controlling the light source to flash N times in one flash window includes: controlling the light source to flash N times in one flash window according to the target level; wherein the plurality of levels are different from one another in one or more of values of N, M, and K, the preset duration, a flash duration of each flash of the light source, and the time interval between adjacent flashes.

In some embodiments, the charging module comprises a power supply input circuit and a voltage acquisition circuit, the power supply input circuit and the voltage acquisition circuit are both connected to the capacitor; and the controlling the charging module to charge the capacitor includes: controlling the power supply input circuit to charge the capacitor; controlling the voltage acquisition circuit to acquire a voltage of the capacitor; and controlling the power supply input circuit to stop charging the capacitor in a case that the voltage of the capacitor is greater than a preset voltage, or controlling the power supply input circuit to stop charging the capacitor after the power supply input circuit has charged the capacitor for a preset duration.

In some embodiments, the pulsed light device further includes a first switch unit, a first terminal of the first switch unit is connected to the light source, and a second terminal of the first switch unit is grounded; and the controlling the light source to flash N times in one flash window includes: controlling the first terminal and the second terminal of the first switch unit to be conducted, to allow the capacitor to discharge to the light source, so as to allow the light source to flash.

In some embodiments, the charging module further includes a second switch unit, and an end of the capacitor connected to the light source is grounded; and the control method further includes: receiving a flashing signal; sending, according to the flashing signal, a charging signal to the charging module and a discharging signal to the first switch unit, to respectively control the first switch unit and the second switch unit to be turned on or off in a preset manner, wherein the first switch unit has an on-off state opposite to that of the second switch unit to realize the following: controlling the light source to flash N times in one flash window, N being an integer greater than or equal to 3; controlling the capacitor to discharge to the light source during each flash of the light source; and controlling the charging module to charge the capacitor during a time interval between two adjacent flashes.

In some embodiments, the pulsed light device further includes a control switch connected to the light source; and the controlling the capacitor to discharge to the light source includes: controlling the control switch to be turned on, to allow the light source to flash.

According to a second aspect, an embodiment of the present disclosure provides a control apparatus for a pulsed light device. The pulsed light device includes a charging module, a capacitor, and a light source. The capacitor is connected between the charging module and the light source, the charging module is configured to charge the capacitor, the capacitor is configured to supply power to the light source to allow the light source to flash. The control apparatus includes a control module, and the control module is configured to control the light source to flash N times in one flash window, N being an integer greater than or equal to 3, and control the capacitor to discharge to the light source during each flash of the light source; and control the charging module to charge the capacitor during a time interval between two adjacent flashes.

According to a third aspect, an embodiment of the present disclosure provides a pulsed light device, including a controller, a charging module, a capacitor, and a light source. The capacitor is connected between the charging module and the light source, the charging module is configured to charge the capacitor, and the capacitor is configured to supply power to the light source to allow the light source to flash. The controller is configured to control the light source to flash N times in one flash window, N being an integer greater than or equal to 3, and control the capacitor to discharge to the light source during each flash of the light source; and control the charging module to charge the capacitor during a time interval between two adjacent flashes.

According to a fourth aspect, an embodiment of the present disclosure provides a service terminal, including a processor and a memory. The processor calls a computer-executable instructions stored in the memory to implement the method according to the first aspect.

According to a fifth aspect, an embodiment of the present disclosure provides a computer-readable storage medium, storing a computer-executable instructions or a computer instruction. The computer-executable instructions or the computer instruction when being executed by a processor implements the method according to according to the first aspect.

According to a sixth aspect, an embodiment of the present disclosure provides a computer-executable instructions product, including computer-executable instruction codes. The computer-executable instruction codes when being executed by a processor implement the method according to according to the first aspect.

The embodiments of the present disclosure provide a control method for a pulsed light device. The pulsed light device includes a charging module, a capacitor, and a light source. The capacitor is connected between the charging module and the light source, the charging module is configured to charge the capacitor, and the capacitor is configured to supply power to the light source to allow the light source to flash. The control method includes: controlling the light source to flash N times in one flash window, N being an integer greater than or equal to 3; controlling the capacitor to discharge to the light source during each flash of the light source; and controlling the charging module to charge the capacitor during a time interval between two adjacent flashes. Compared to releasing all the energy stored in the capacitor at once, the method according to the present disclosure, by controlling the light source to flash N times in one flash window, wherein N is an integer greater than or equal to 3, so as to enable the pulsed light device to release the energy in the capacitor by flashing at least three flashes, to split the single discharging process of the capacitor into multiple charging and discharging processes, and to control the energy of each discharge by means of a segmented approach, to avoid the high-energy impact brought by a single discharge, such that the pulsed light device can decentralize the energy and can be used to treat skin in a more gentle and controllable manner, without sacrificing the required intensity of the pulsed light. This not only reduces the pain and other discomforts of users during the treatment process but also minimizes the impact on sensitive components in the device, thereby prolonging the service life of the device. Moreover, charging the capacitor immediately after each discharge can increase the amount of energy released by the pulsed light device during the flash window, thereby enhancing the effect on the skin.

### BRIEF DESCRIPTION OF THE DRAWINGS

To illustrate the technical solutions in the embodiments of the present disclosure more clearly, the following briefly introduces the accompanying drawings required for describing the embodiments of the present disclosure. Apparently, the accompanying drawings in the following description are only some embodiments of the present disclosure, and those skilled in the art may obtain other drawings based on these accompanying drawings without creative efforts.

FIG. 1 is a schematic diagram of a pulsed light device according to an embodiment of the present disclosure.

FIG. 2 is a schematic flow diagram of a control method for a pulsed light device according to an embodiment of the present disclosure.

FIG. 3 is a schematic flow diagram of a control method for a pulsed light device according to another embodiment of the present disclosure.

FIG. 4 is a schematic diagram of a pulsed light device according to another embodiment of the present disclosure.

FIG. 5 is a schematic diagram of a charging module according to an embodiment of the present disclosure.

FIG. 6 is a schematic diagram of a charging module according to another embodiment of the present disclosure.

FIG. 7 is a schematic diagram of a charging module according to still another embodiment of the present disclosure.

FIG. 8 is a schematic diagram of a power supply input circuit according to an embodiment of the present disclosure.

FIG. 9 is a schematic diagram of a voltage acquisition circuit according to an embodiment of the present disclosure.

FIG. 10 is a schematic diagram of a power conversion circuit according to an embodiment of the present disclosure.

FIG. 11 is a schematic diagram of a voltage regulator circuit according to an embodiment of the present disclosure.

FIG. 12 is a schematic diagram of a pulsed light device according to still another embodiment of the present disclosure.

FIG. 13 is a schematic diagram of a voltage conversion circuit according to an embodiment of the present disclosure.

FIG. 14 is a schematic diagram of a pulsed light device according to still another embodiment of the present disclosure.

FIG. 15 is a schematic diagram of a driving circuit according to an embodiment of the present disclosure.

FIG. 16 is a schematic diagram of a charging module according to still another embodiment of the present disclosure.

FIG. 17 is a schematic diagram of a pulsed light device according to still another embodiment of the present disclosure.

FIG. 18 is a schematic diagram of a pulsed light device according to still another embodiment of the present disclosure,

FIG. 19 is a schematic diagram of a pulsed light device according to still another embodiment of the present disclosure.

### DETAILED DESCRIPTION OF THE EMBODIMENTS

In order to enable persons skilled in the art to better understand the present disclosure, the following clearly and completely describes the technical solutions in the embodiments of the present disclosure with reference to the accompanying drawings in the embodiments of the present disclosure.

The embodiments of the present disclosure provide a control method for a pulsed light device and a related device, which can control the light source to flash multiple times, so as to disperse the energy, thereby improving the user experience and prolonging the service life of the device.

In order to better understand the embodiments of the present disclosure, the related art is described.

Pulsed light technology has been widely applied in the fields of beauty and medicine. The pulsed light device emits pulsed light of a specific wavelength to treat skin, to achieve various effects such as spot removal and hair removal.

The pulsed light device in the related art typically uses a single discharge to generate the desired intense pulsed light. Although the single discharge can provide high-intensity pulsed light in a short period, the single high-intensity discharge will reduce the user's sense of use. Additionally, it will also put pressure on the sensitive components inside the device, thereby affecting the stability and service life of the device.

In order to solve the above technical problem, the embodiments of the present disclosure provide a control method for a pulsed light device. The pulsed light device includes a charging module, a capacitor, and a light source. The capacitor is connected between the charging module and the light source, the charging module is configured to charge the capacitor, and the capacitor is configured to supply power to the light source to allow the light source to flash. The control method includes: controlling the light source to flash N times in one cycle (flash window), N being an integer greater than or equal to 3; controlling the capacitor to discharge to the light source during each flash of the light source; and controlling the charging module to charge the capacitor during a time interval between two adjacent flashes. Compared to releasing all the energy stored in the capacitor at once, the method in the present disclosure, by controlling the light source to flash N times in one cycle (flash window), wherein N is an integer greater than or equal to 3, so as to enable the pulsed light device to release the energy in the capacitor by flashing at least three flashes, to split the single discharging process of the capacitor into multiple charging and discharging processes, and to control the energy of each discharge by means of a segmented approach, to avoid the high-energy impact brought by a single discharge, such that the pulsed light device can decentralize the energy and can be used to treat skin in a more gentle and controllable manner, without sacrificing the required intensity of the pulsed light. This not only reduces the pain and other discomforts of users during the treatment process but also minimizes the impact on sensitive components in the device, thereby prolonging the service life of the device. Moreover, charging the capacitor immediately after each discharge can increase the amount of energy released by the pulsed light device during the cycle (flash window), thereby enhancing the effect on the skin.

To better understand the embodiments of the present disclosure, the structure of the pulsed light device is described below.

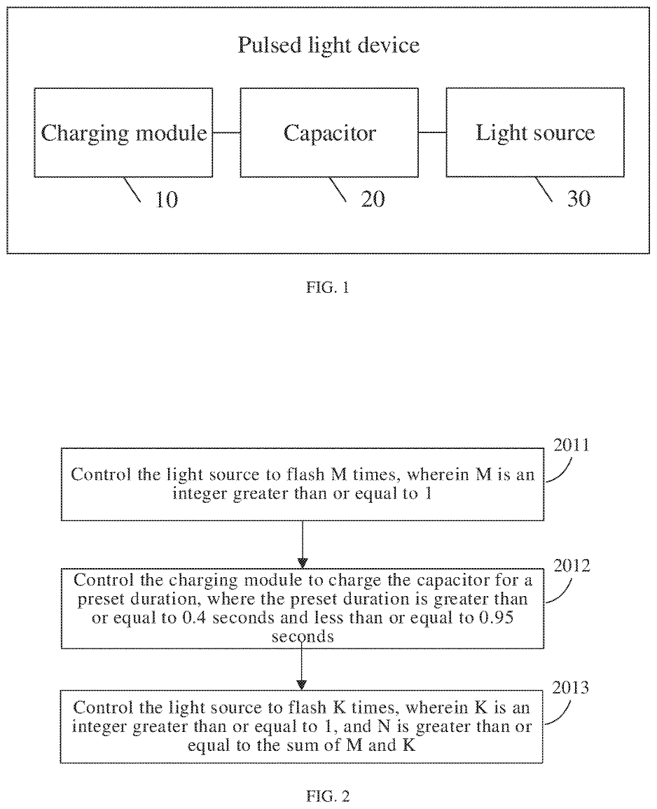

Referring to FIG. 1, FIG. 1 is a schematic diagram of a pulsed light device according to an embodiment of the present disclosure. As shown in FIG. 1, the pulsed light device may include a charging module 10, a capacitor 20, and a light source 30. The capacitor 20 is connected between the charging module 10 and the light source 30.

In the present disclosure, the pulsed light device may be a hair removal device or a skin rejuvenation device, etc., and the following mainly uses a hair removal device as an example for illustration.

The control method in the present disclosure may be applied to a controller of a pulsed light device. The pulsed light device may include a charging module, a capacitor, and a light source. The capacitor is connected between the charging module and the light source. The charging module may charge the capacitor, and the capacitor may supply power to the light source, to allow the light source to flash. The control method may include the following steps.

Step 201, the light source is controlled to flash N times in one flash window, where N is an integer greater than or equal to 3.

The capacitor is controlled to discharge to the light source during each flash of the light source.

Besides, the charging module is controlled to charge the capacitor during a time interval between two adjacent flashes.

The light source may flash a plurality of times by way of a plurality of operations of the operator. The light source is controlled to flash N times in one cycle (namely one flash window) is distinct from the plurality of times of flash brought by plurality of operations. To avoid ambiguity, in the embodiments of the present disclosure, the aforementioned “cycle”is also referred to as the “flash window”.

In one flash window, the controller may control the capacitor to discharge so that the light source sequentially generates a first pulsed light, a second pulsed light, . . . , a Nth pulsed light for irradiating a user's skin.

The flash window may be understood as a time window. The working time of the pulsed light device may include one flash window or a plurality of flash windows. In the case that the working time of the pulsed light device includes a plurality of flash windows, the pulsed light device may have the same or different working modes in different flash windows. The following embodiments mainly use the pulsed light device having the same working mode in different flash windows as an example for illustration.

In some embodiments, the pulsed light device may include a user-operable switch. In response to a user's trigger operation of the switch, the pulsed light device may work in a single flash window, or work periodically with the flash window as a cycle.

Illustratively, the pulsed light device may include a push-button switch disposed on a side of the body of the pulsed light device. A short press of the switch may allow the pulsed light device to work in a single flash window; and a long press of the switch may allow the pulsed light device to work periodically with the flash window as a cycle.

For example, the user may give a short press to the switch, such that the light source flashes N times in one flash window. After completing the N flashes, the pulsed light device stops flashing until the user triggers the switch again.

For another example, the user may give a long press to the switch, such that the light source flashes N times in one flash window, and after a preset time interval, the light source continues to flash N times in the next flash window, and then after another preset time interval, the light source continues to flash N times in the subsequent flash window,. and so on, until the user stops triggering the switch. The preset time interval is a time duration between the Nth pulsed light in one flash window and the first pulsed light in the adjacent subsequent flash window.

In one flash window, the controller may first control the capacitor to discharge, to allow the light source to generate the first pulsed light irradiating the user's skin. After irradiation of the first pulsed light has stopped for a first preset duration, the controller may control the capacitor to discharge again, to allow the light source to generate the second pulsed light irradiating the user's skin. After irradiation of the second pulsed light has stopped for a second preset duration, the controller may control the capacitor to discharge once more, to allow the light source to generate the Nth pulsed light irradiating the user's skin.

In one flash window, after generating the first pulsed light that irradiates the user's skin by the light source, the pulsed light device waits for the first preset duration rather than immediately controls the light source to generate the second pulsed light that irradiates the user's skin. In other words, after the irradiation of the first pulsed light has stopped for the first preset duration, the pulsed light device controls the light source to generate the second pulsed light that irradiates the user's skin. Similarly, after generating the second pulsed light that irradiates the user's skin by the light source, the pulsed light device waits for the second preset duration rather than immediately controls the light source to generate the third pulsed light that irradiates the user's skin. In other words, after the irradiation of the second pulsed light has stopped for the second preset duration, the pulsed light device controls the light source to generate the third pulsed light that irradiates the user's skin. This can reduce the discomfort that users may experience due to high energy levels and also avoid the continuous emission of the pulsed light from the light source onto the user's skin, which could otherwise cause the skin temperature to become too high and result in damage.

In some embodiments, the pulsed light device may further include a timer. When stopping the irradiation of the first pulsed light, the pulsed light device may activate the timer to start timing. After the timer has worked for the first preset duration, the pulsed light device may control the light source to emit the second pulsed light irradiating the user's skin. Similarly, when stopping the irradiation of the second pulsed light, the pulsed light device may activate the timer to start timing again. After the timer has worked for the second preset duration, the pulsed light device may control the light source to emit the third pulsed light irradiating the user's skin.

The pulsed light device may control the light source to flash N times in one flash window based on a preset control logic, to precisely control the number of flashes of the light source (e.g., N times), so as to meet different user requirements.

Each flash of the light source may output a particular amount of energy to achieve skincare. The total energy released during the flash window may be adjusted by controlling the number of flashes (N) of the light source in each flash window. Each flash of the light source releases a certain amount of energy to the skin, and the number of flashes in one flash window determines the total energy released to the skin. By releasing energy to the skin at least 3 times in a flash window, the skin can be treated by a superposed and accumulated energy, which can avoid excessively high energy released in a single discharge, thereby allowing for a more gentle and controllable treatment of the skin. This reduces pain and other discomforts of the user during using the device and also reduces the impact on sensitive components in the device, thereby prolonging the service life of the device.

Each flash of the light source requires a certain amount of energy. By controlling the capacitor to discharge to the light source, the electrical energy stored in the capacitor can be converted into light energy to provide to the light source to flash, even if the energy in the capacitor is output by the light source of the pulsed light device in the form of pulsed light.

By controlling the capacitor to perform one discharge to the light source to achieve one flash, the light source of the present disclosure can realize one flash each time the capacitor discharges. The duration of each flash of the light source can be controlled by precisely controlling the duration of discharge of the capacitor to the light source, so that the capacitor only releases enough electrical energy to produce the required light intensity and duration, rather than completely depleting the capacitor. This can save energy and ensure that the light source is able to maintain a stable output during the successive flashing process.

In one application scenario, to achieve long-term or even permanent hair removal effects, N may be set to 6. Continuous high-frequency flash can effectively damage the hair follicles to achieve the long-term or even permanent hair removal effect. By using a continuous flashing mode and precisely controlling the energy output of each flash, the safety and treatment effect can be ensured, and the damage to the surrounding skin is reduced.

In another application scenario, for users who are undergoing pulsed light hair removal treatment for the first time or have sensitive skin, in order to enhance the experience of the hair removal treatment, N may be set to 4. By reducing the number of flashes, the risk of damage to the skin due to excessive heat energy can be lowered. A smaller number of flashes (N=4) indicates that the total energy absorbed by the skin in one flash window is lower, which can reduce the discomfort in the hair removal process, such as heat sensation, stinging, or swelling, thereby improving user comfort.

Each flash of the light source requires the capacitor to discharge to the light source, thus consuming the electrical energy stored in the capacitor. After the discharge, the amount of power in the capacitor decreases. To ensure that the energy or duration of the next discharge can meet a preset requirement, the capacitor needs to be recharged to ensure that there is enough energy for the next flash of the light source. Therefore, during the time interval between two adjacent flashes of the light source, the charging module may be controlled to charge the capacitor to replenish energy for the next flash of the light source. Charging and discharging based on the preset logic ensures that the capacitor is not charged and discharged simultaneously, and also guarantees that the capacitor can be replenished before the next flash, thereby maintaining the continuous operation and efficiency of the pulsed light device. As such, the energy released by the pulsed light device during the flash window can be increased by charging the capacitor immediately after each discharge, thus the effect on the skin is improved.

In some embodiments, the aforementioned steps form the basic working flash window of the pulsed light device: discharge-charge-discharge-charge. This flash window ensures that the pulsed light device continuously and stably outputs the pulsed light of a predetermined intensity and number of times throughout the process.

It should be noted that, since the energy in the capacitor is not completely exhausted after each discharge, the charging module does not need to charge the capacitor from zero, which can shorten the charging duration and improve the efficiency of the entire process. Moreover, when charging the capacitor, the capacitor only needs to be charged based on a preset charging logic, without being fully charged. However, it is necessary to meet the energy required for the next flash of the light source, to not only ensure that the capacitor will not be overcharged, but also ensure that the capacitor is sufficiently charged before the next flash, so as to maintain the continuous operation of the pulsed light device.

The length of the time interval may be adjusted according to a specific application of the pulsed light device and a flash requirement of the light source, to ensure that the light intensity and duration meet the predetermined requirement.

Compared to releasing all the energy stored in the capacitor at once, the method according to the present disclosure, by controlling the light source to flash N times in one flash window, wherein N is an integer greater than or equal to 3, so as to enable the pulsed light device to release the energy in the capacitor by flashing at least three flashes, to split the single discharging process of the capacitor into multiple charging and discharging processes, and to control the energy of each discharge by means of a segmented approach, to avoid the high-energy impact brought by a single discharge, such that the pulsed light device can decentralize the energy and can be used to treat skin in a more gentle and controllable manner, without sacrificing the required intensity of the pulsed light. This not only reduces the pain and other discomforts of users during the treatment process but also minimizes the impact on sensitive components in the device, thereby prolonging the service life of the device. Moreover, charging the capacitor immediately after each discharge can increase the amount of energy released by the pulsed light device during the flash window, thereby enhancing the effect on the skin.

It will be understood that in particular embodiments, the time interval between two adjacent flashes may be different or the same.

Based on the research of the charge and discharge principle of the capacitor and the research on the characteristics of skin's absorption of energy, it is found that in each flash window, by controlling the light source to flash in multiple stages and making a longer time interval between adjacent stages, for example, the time interval is ranged from 0.4 seconds to 0.95 seconds (optionally from 0.5 seconds to 1.8 seconds), the capacitor can be charged more. This allows the capacitor's power is restored to a certain level to meet the subsequent discharge requirement, and also avoids interruptions caused by too long charging time.

The following provides an example to illustrate the multi-stage flashing.

In some embodiments, referring to FIG. 2, FIG. 2 is a schematic flow diagram of a control method according to an embodiment of the present disclosure. Step 201 may include;

Step 2011, the light source is controlled to flash M times, where M is an integer greater than or equal to 1.

In a first stage, the light source may be controlled to flash M times, which lays the foundation for the flashing in a second stage. For example, in the hair removal treatment, the M flashes in the first stage can soften the hair follicles and achieve preliminary hair removal, or the M flashes are preparations for subsequent hair removal. And, by setting different values of M, it is possible to control the preliminary flashing of the light source for different user requirements.

It can be understood that in a configuration that M is greater than 1, the charging module may be controlled to charge the capacitor during a time interval between two adjacent flashes in the M flashes.

Step 2012, the charging module is controlled to charge the capacitor for a preset duration, where the preset duration is greater than or equal to 0.4 seconds and less than or equal to 0.95 seconds.

After the M flashes in the first stage, the charging module may be controlled to charge the capacitor, and the charging duration may be within a range from 0.4 seconds to 0.95 seconds. The capacitor can be charged to restore the power to a certain level in the charging duration, to meet the subsequent discharge requirement, and also to avoid interruption caused by too long charging time.

Step 2013, the light source is controlled to flash K times, where K is an integer greater than or equal to 1, and N is greater than or equal to the sum of M and K.

After the charging to the capacitor is completed, the light source may be controlled to flash for the second stage, namely, the light source is controlled to flash K times, to further enhance the treatment effect of the flash. For example, in the hair removal treatment, the M flashes in the first stage can soften the hair follicles, which provides a preparation for the subsequent hair removal. Based on this, the K flashes in the second stage can deeply act on the hair follicles so as to effectively destroy the hair follicle structure, thereby inhibiting or even permanently preventing the regrowth of the hair. In the second stage, the number of flashes of the light source may be adjusted to adapt to different user requirements.

Illustratively, the preset duration may be 0.4 seconds, 0.5 seconds, 0.55 seconds, 0.65 seconds, 0.75 seconds, 0.85 seconds or 0.95 seconds. It should be understood that the above is an exemplary description of the preset duration and is not to limit the specific value of the preset duration. For example, the preset duration may be 0.45 seconds.

In a configuration that the preset duration is 0.4 seconds, the charging module charges the capacitor for the shortest duration, which can maintain a fast flash rhythm and ensure that the capacitor has sufficient energy for effective flash. This configuration is suitable for applications that require rapid consecutive flashing.

In a configuration that the preset duration is 0.5 seconds, the charging module provides a relatively short charging duration for the capacitor, which is suitable for applications that require a faster flashing rhythm. For example, this configuration may be used for light to moderate hair removal treatments, which ensures that the capacitor receives sufficient energy for each effective flash while maintaining efficiency.

In a configuration that the preset duration is 0.55 seconds, the charging duration is slightly prolonged, and the capacitor is allowed to obtain more energy replenishment during the interval between the first stage and the second stage. For example, this configuration is suitable for moderate-intensity skin treatments, such as moderate hair removal, skin refinement, and improvement of minor pigmentation issues.

In a configuration that the preset duration period is 0.65 seconds, more charging duration is provided for the capacitor, such that the capacitor releases more energy during the discharge process in the second stage. For example, this configuration may be suitable for deep skin treatments, including deep hair removal, significant skin texture improvement, and collagen rebuilding.

In a configuration that the preset duration is 0.75 seconds, the light source is allowed to flash with a high energy to achieve depth and intensity applications, such as hair removal for high-density hair.

In a configuration that the preset duration is 0.85 seconds, the capacitor can obtain a higher amount of energy reserve before the flash of the second stage, thereby providing sufficient preparation time for some special high-intensity treatments, such as deep skin remodeling, removal of deep skin scars, or deep hair removal for large areas.

In a configuration that the preset duration is 0.95 seconds, the charging module charges the capacitor for the longest time, which allows for a high-energy output in the second stage, supporting strong and deep treatment effects.

In some embodiments, the preset duration is greater than or equal to 0.5 seconds and less than or equal to 0.7 seconds.

After controlling the light source to flash M times, the charging module may be controlled to charge the capacitor for the preset duration, where the preset duration may be greater than or equal to 0.5 seconds and less than or equal to 0.7 seconds, and then the light source may be controlled to flash K times.

In some application scenarios, a two-stage flash may be provided, and in this case, N is equal to the sum of M and K. Examples are provided below for illustration.

Illustratively, to perform hair removal treatment, N is set to be equal to the sum of M and K, e.g., N(4)=M(2)+K(2), and the preset duration is 0.6 seconds. The pulsed light device may control the light source to emit two flashes in the first stage to pre-treat the target hair follicles, thereby heating the follicle area. This increases the temperature of the follicle area, to make the hair follicle structure slightly fragile, providing a preparation for the following hair removal. After completing the two flashes, the charging module is controlled to charge the capacitor for 0.6 seconds, to ensure that the capacitor obtains sufficient energy replenishment in a short time, thereby preparing for the second stage of hair removal. Based on the pre-treatment of the first stage, the light source may be controlled to emit two more flashes in the second stage to further damage the hair follicles. Utilizing the thermal effect accumulated from the first two flashes, the subsequent two flashes can more effectively penetrate the hair follicles to destroy their growth ability, thereby inhibiting or preventing the regrowth of hair.

Illustratively, to perform hair removal treatment, N is set to be equal to the sum of M and K, e.g., N(5)=M(3)+K(2), and the preset duration is 0.5 seconds. The pulsed light device may control the light source to emit three flashes in the first stage to pre-treat the target hair follicles, thereby heating the follicle area. This increases the temperature of the follicle area, to make the hair follicle structure slightly fragile, thereby preparing for the following hair removal. After completing the three flashes, the charging module is controlled to charge the capacitor for 0.5 seconds, to ensure that the capacitor obtains sufficient energy replenishment in a short time, thereby preparing for the second stage of hair removal. Based on the pre-treatment of the first stage, the light source may be controlled to emit two more flashes in the second stage to further damage the hair follicles. Utilizing the thermal effect accumulated from the first three flashes, the subsequent two flashes can more effectively penetrate the hair follicles to destroy their growth ability, thus inhibiting or preventing the regrowth of hair.

In some embodiments, the flashes in the second stage have higher energy than the flashes in the first stage, to ensure that the energy penetrates deep into the hair follicles to effectively damage the structure of the hair follicles.

In some other application scenarios, N is greater than the sum of M and K.

That is to say, a multi-stage flashing may be performed in one flash window, where the number of stages is greater than or equal to 2. The time interval between adjacent stages is greater than or equal to 0.4 seconds and less than or equal to 0.95 seconds, and capacitor is charged during this time interval to replenish energy. For example, after step 2013, the method further includes step 2014: controlling the charging module to charge the capacitor for a preset duration, where the preset duration is greater than or equal to 0.4 seconds and less than or equal to 0.95 seconds; step 2015, controlling the light source to flash L times, where L is an integer greater than or equal to 1, and N is greater than or equal to the sum of M, K, and L.

Illustratively, to perform hair removal treatment, N is set to be equal to the sum of M, K, and L, e.g., N(6)=M(2)+K(2)+L(2), and the preset duration is 0.7 seconds, where L represents the number of flashes in a third stage after the first and second stages in one flash window. The pulsed light device may control the light source to emit two flashes in the first stage. After completing the two flashes, the capacitor is controlled to be charged for 0.7 seconds. Then, based on the pre-treatment in the first stage, the light source is controlled to emit two more flashes in the second stage. After that, the capacitor is controlled to be charged for another 0.7 seconds, and the light source is controlled to emit two more flashes. As a result, the total number of flashes is 6. As such, by treating the skin in batches of three stages, the photothermal effect is enhanced through the additional number of flashes L, thereby providing enhanced treatment to target hair follicles. Utilizing the thermal effect accumulated in the first two stages, the additional two flashes can more effectively penetrate the hair follicles to destroy their growth ability, thus inhibiting or preventing the regrowth of hair, In the foregoing embodiments of the multi-stage flashing control, the values of M, K, the preset duration, and/or L are described in detail below.

In some embodiments, M is greater than 1.

In a configuration that M is greater than 1, the time interval between adjacent flashes in the M flashes may be greater than or equal to 0.06 seconds and less than or equal to 0.4 seconds.

In the configuration that M is greater than 1, that is, the light source is controlled to flash multiple times in the first stage, the time interval between adjacent flashes may be greater than or equal to 0.06 seconds and less than or equal to 0.4 seconds, for example, 0.06 seconds, 0.08 seconds, 0.1 seconds, 0.12 seconds, 0.15 seconds, 0.17 seconds, 0.2 seconds, 0.24 seconds, 0.26 seconds, 0.3 seconds, 0.34 seconds, 0.38 seconds, or 0.4 seconds. For example, in a configuration that M is equal to 2, the light source is controlled to flash twice, and the time interval between the two flashes is precisely controlled to be between 0.06 seconds and 0.4 seconds.

In this way, multiple flashes are applied to the skin in the first stage, and the time interval between adjacent flashes in this stage is controlled between 0.06 seconds and 0.4 seconds, which not only helps to avoid skin irritation but also allows for more energy replenishment, ensuring a better cumulative energy effect.

In some embodiments, in the configuration that M is greater than 1, the time interval between adjacent flashes in the M flashes is greater than or equal to 0.1 seconds and less than or equal to 0.3 seconds, to improve operability and ensure a cumulative energy effect.

In some embodiments, the preset duration is greater than or equal to 0.5 seconds and less than or equal to 0.8 seconds.

After controlling the light source to flash M times, the charging module may be controlled to charge the capacitor for the preset duration, where the preset duration may be greater than or equal to 0.5 seconds and less than or equal to 0.8 seconds, and then the light source may be controlled to flash K times.

In some embodiments, in a configuration that K is greater than 1, the time interval between adjacent flashes in the K flashes is greater than or equal to 0.06 seconds and less than or equal to 0.4 seconds, such as 0.06 seconds, 0.08 seconds, 0.1 seconds, 0.12 seconds, 0.15 seconds, 0.17 seconds, 0.2 seconds, 0.24 seconds, 0.26 seconds, 0.3 seconds, 0.34 seconds, 0.38 seconds, or 0.4 seconds.

Similarly, in the configuration that K is greater than 1, that is, the light source is controlled to flash multiple times in the second stage, the time interval between adjacent flashes may be greater than or equal to 0.06 seconds and less than or equal to 0.4 seconds, for example, 0.06 seconds, 0.08 seconds, 0.1 seconds, 0.12 seconds, 0.15 seconds, 0.17 seconds, 0.2 seconds, 0.24 seconds, 0.26 seconds, 0.3 seconds, 0.34 seconds, 0.38 seconds, or 0.4 seconds. For example, in a configuration that K is 3, the light source is controlled to flash three times, and the time interval between adjacent flashes is precisely controlled to be between 0.06 seconds and 0.4 seconds.

In some embodiments, in the configuration that K is greater than 1, the time interval between adjacent flashes in the K flashes is greater than or equal to 0.1 seconds and less than or equal to 0.3 seconds, to improve operability and ensure a cumulative energy effect.

It should be noted that different embodiments may be combined and adjusted according to different user requirements and objectives. For example, the values of M, K, and N, as well as the corresponding time interval and the preset charging duration, may be flexibly adjusted according to the size of the target area, the depth of treatment, and/or the skin type, to improve the flexibility and applicability of the pulsed light device.

In an application scenario, M is equal to 3, the time interval between adjacent flashes in the M flashes is 0.08 seconds, K is equal to 4, the time interval between adjacent flashes in the K flashes is 0.35 seconds, and the preset duration is 0.6 seconds. That is, the light source is first controlled to flash 3 times, and the time interval between adjacent flashes is precisely controlled to 0.08 seconds, such that the initial heat treatment can be performed on the target area quickly and continuously. After the 3 flashes are completed, the charging module starts immediately to charge the capacitor for 0.6 seconds, such that the capacitor can quickly restore energy and provide necessary electric energy for the K flashes in the next stage. After the charging of the capacitor is completed, the light source is controlled to flash 4 times, and the time interval between adjacent flashes is adjusted to 0.35 seconds.

By refining the time intervals between adjacent flashes in the M flashes and the K flashes, it is possible to avoid rapid heat accumulation due to excessively short intervals, while maintaining sufficient thermal effects. For example, M is equal to 2, the time interval between adjacent flashes in the M flashes is 0.2 seconds, K is equal to 3, the time interval between adjacent flashes in the K flashes is 0.2 seconds, and the preset duration is 0.6 seconds. The light source is controlled to flash twice first, and the interval between the two flashes is 0.2 seconds, which can avoid the heat accumulation caused by an excessively short time interval, reduce the potential damage to the skin, and ensure that there is enough thermal effect to begin acting on the target area. After that, the charging module is controlled to charge the capacitor for 0.6 seconds to provide the required energy for the next three flashes of the light source. After the charging of the capacitor is completed, the light source is controlled to continue to flash three times, with a time interval of 0.2 seconds between adjacent flashes in the three flashes. This can further deepen the treatment of the target area, enhancing the effectiveness. In the embodiments, precise management is achieved, thus improving the user experience and the safety of the device.

The flash duration of each flash of the light source will be described below. In the embodiments of the present disclosure, the flash duration refers to the pulse width of the electrical signal that drives the light source to flash, i.e., the duration of the current or voltage pulse.

In some embodiments, the flash duration of each flash of the light source is greater than or equal to 0.3 milliseconds and less than or equal to 10 milliseconds per flash, for example, 0.3 milliseconds, 0.4 milliseconds, 0.65 milliseconds, 0.8 milliseconds, 0.95 milliseconds, 1 millisecond, 1.5 milliseconds, 2 milliseconds, 2.5 milliseconds, 3 milliseconds, 3.5 milliseconds, 4 milliseconds, 4.5 milliseconds, 5 milliseconds, 5.5 milliseconds, 6 milliseconds, 6.5 milliseconds, 7 milliseconds, 7.5 milliseconds, 8 milliseconds, 8.5 milliseconds, 9 milliseconds, 9.5 milliseconds, or 10 milliseconds.

The flash duration of the light source each time is set between 0.3 milliseconds and 10 milliseconds, which provides great flexibility for the pulsed light device to allow it to precisely treat different types of skin and hair. A shorter flash duration (close to 0.3 milliseconds) may be suitable for treatments that require fine control, such as hair removal for sensitive skin. Using a shorter flash duration may precisely heat the hair follicles, which reduces unnecessary heating of the deeper skin and avoiding excessive heating of the target tissue. A longer flash duration (close to 10 milliseconds) may be suitable for deep hair removal, such as hair removal for thick hair. Thick hair may absorb more energy, and a longer flash duration can ensure that sufficient heat is transferred deep into the hair follicle, effectively damaging the hair follicle structure and achieving hair removal effects. A longer flash duration can provide a more pronounced thermal effect to achieve better results.

Moreover, by setting the flash duration of the light source between 0.3 milliseconds and 10 milliseconds, on the one hand, it avoids the high-energy impact brought by excessively long single flash duration, and on the other hand, it also prevents the ineffectiveness caused by an excessively short single flash duration.

In some embodiments, the flash duration of each flash of the light source is greater than or equal to 0.5 milliseconds and less than or equal to 4 milliseconds, such as 0.5 milliseconds, 0.6 milliseconds, 0.65 milliseconds, 0.8 milliseconds, 0.95 milliseconds, 1 millisecond, 1.5 milliseconds, 2 milliseconds, 2.5 milliseconds, 3 milliseconds, 3.5 milliseconds, or 4 milliseconds.

By setting the flash duration of the light source between 0.5 milliseconds and 4 milliseconds, the long duration of a single flash can be avoided, which can prevent the skin from being exposed to high energy for a long time, and can reduce the risk of skin damage due to heat accumulation. This is particularly important for sensitive skin, and improves safety. Moreover, it can also ensure that a single flash can also generate sufficient thermal effect to achieve better results.

In some embodiments, in step 2011, in a configuration that M is equal to 2, a flash duration of a first flash is equal to or less than a flash duration of a second flash.

In the configuration that M is equal to 2, that is, the light source is controlled to flash twice in the first stage, the flash duration of the first flash may be equal to the duration of the second flash, such a setting may be suitable for a uniform treatment on target areas. For example, in the hair removal treatment, if the hair density in the target area is relatively uniform, two flashes of equal flash duration can ensure that each hair follicle receives the same amount of thermal energy, thus achieving a uniform hair removal effect.

In the configuration that M is equal to 2, the flash duration of the first flash may be less than the duration of the second flash, to gradually enhance the treatment effect. For example, after an initial mild thermal effect, the second longer-duration flash can penetrate deeper into the hair follicles or the deeper layers of the skin, increasing the intensity. Such a setting may be suitable for a skin treatment of superficial treatment first and then deeper treatment.

In some embodiments, in step 2013, in the configuration that K is equal to 2, a flash duration of a first flash is equal to or less than a flash duration of a second flash.

In the configuration that K is equal to 2, that is, the light source is controlled to flash twice in the second stage, the flash duration of the first flash may be equal to the flash duration of the second flash, and such a setting may be suitable for a uniform treatment on target areas. For example, in the hair removal treatment, if the hair density in the target area is relatively uniform, two flashes with equal flash duration can ensure that each hair follicle receives the same amount of thermal energy, thus achieving a uniform hair removal effect.

In the configuration that K is equal to 2, the flash duration of the first flash may be less than the flash duration of the second flash, which can gradually enhance the effect. For example, after an initial mild thermal effect, the second longer-duration flash can penetrate deeper into the hair follicles or the deeper layers of the skin, increasing the intensity. Such a setting may be suitable for a skin treatment of superficial treatment first and then deeper treatment.

By flexibly setting the flash duration, the pulsed light device can meet the different needs of users and improve user experience.

In some embodiments, in step 2013, in the configuration that K is equal to 2, the flash duration of the first flash is less than the flash duration of the second flash. Moreover, the flash duration of the first flash is greater than or equal to 0.3 milliseconds and less than or equal to 0.7 milliseconds, and the flash duration of the second flash is greater than or equal to 1.2 milliseconds and less than or equal to 3 milliseconds.

In the configuration that K is equal to 2, that is, the light source is controlled to flash twice in the second stage, the light source is controlled to flash for a duration greater than or equal to 0.3 milliseconds and less than or equal to 0.7 milliseconds for the first flash, which allows for a more precise initial thermal effect on the target area with a shorter flash duration, to soften the target area; the light source is controlled to flash for a duration greater than or equal to 1.2 milliseconds and less than or equal to 3 milliseconds for the second flash, which is significantly longer than the duration of the first flash. This longer flash duration allows for a deeper effect on the target tissue, achieving a better treatment effect through accumulated heat.

The combination of the first shorter duration flash and the second longer duration flash not only optimizes the effect, but also reduces potential skin damage through gradual heating. Moreover, a better treatment effect can be achieved through the progressive accumulation of energy.

In some embodiments, the energy emitted by the light source flash is positively correlated with the voltage of the capacitor. The capacitor supplies the electrical energy it stores for the light source to flash. The higher the voltage of the capacitor, the more electrical energy it can supply to the light source, thereby generating stronger or more pulsed light. By increasing the voltage of the capacitor, the energy of the flash of the light source can be enhanced, thus making the photothermal effect during the treatment process more significant.

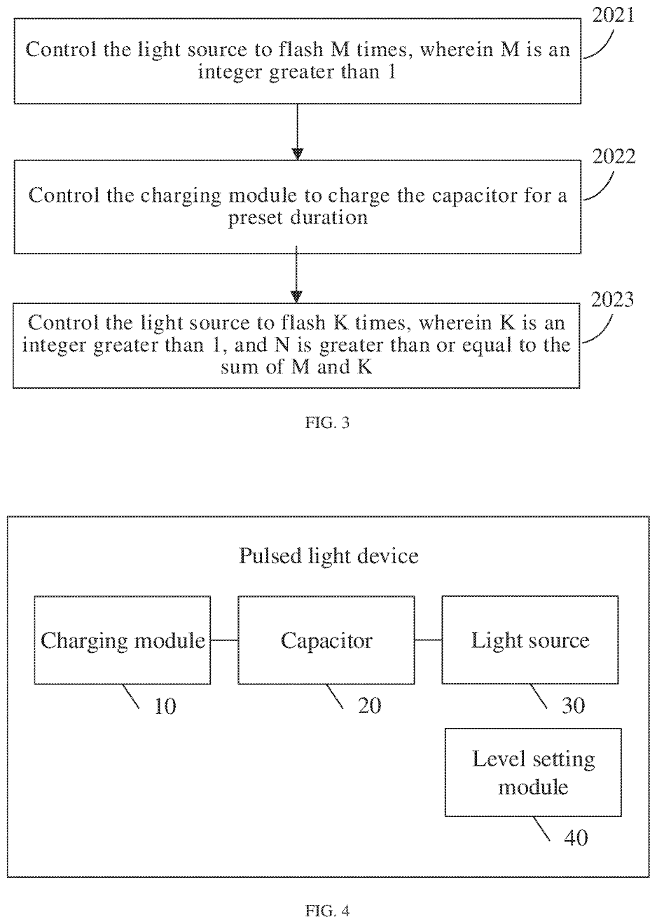

In some embodiments, referring to FIG. 3, FIG. 3 is a schematic flow diagram of a control method according to another embodiment of the present disclosure. Step 201 may include:

Step 2021, the light source is controlled to flash M times, where M is an integer greater than 1.

In a first stage, the light source may be controlled to flash M times, which lays the foundation for the flashing in a second stage. For example, in the hair removal treatment, the M flashes in the first stage can soften the hair follicles and achieve preliminary hair removal, or provide a preparation for subsequent hair removal. The light source may be controlled to flash to meet different user needs by setting different values of M.

For the sake of convenience and brevity in description, the implementation of step 2021 may refer to the step 2011, which is not detailed herein.

Step 2022, the charging module is controlled to charge the capacitor for a preset duration.

After the M flashes in the first stage, the charging module may be controlled to charge the capacitor.

For the sake of convenience and brevity in description, the implementation of step 2022 may refer to the step 2012, which is not detailed herein.

Step 2023, the light source is controlled to flash K times, where K is an integer greater than 1, and N is greater than or equal to the sum of M and K.

After the charging of the capacitor is completed, the light source may be controlled to flash in the second stage. That is, the light source may be controlled to flash K times, to further enhance the treatment effect the flashing of the light source.

For the sake of convenience and brevity in description, the implementation of step 2023 may refer to the step 2013, which is not detailed herein.

In some embodiments, the preset duration is greater than or equal to 0.4 seconds and less than or equal to 0.95 seconds.

Illustratively, the preset duration may be 0.4 seconds, 0.5 seconds, 0.55 seconds, 0.65 seconds, 0.75 seconds, 0.85 seconds, or 0.95 seconds. It should be understood that the above is an exemplary description of the preset duration and not to limit the value of the preset duration. For example, the preset duration may be 0.45 seconds.

In some embodiments, the pulsed light device may be a hair removal device or a skin rejuvenation device.

In a configuration that the pulsed light device is a hair removal device, the hair removal device may utilize particular flash duration to effectively remove hair of different depths and thicknesses. A shorter flash duration (close to 0.3 milliseconds) is suitable for fine hair, while a longer flash duration (close to 10 milliseconds) is suitable for coarser hair, ensuring that the light energy penetrates deeply into the hair follicles to sufficiently disrupt their structures.

In a configuration that the pulsed light device is a skin rejuvenation device, the skin rejuvenation device may adjust the flash duration to provide precise skin rejuvenation treatment. A shorter flash duration (close to 0.3 milliseconds) is suitable for subtle improvements to the superficial layer of the skin, such as enhancing skin texture. A longer flash duration (close to 10 milliseconds) may penetrate deeper into the skin, achieving a deep skin rejuvenation effect.

FIG. 4 is a schematic diagram of a pulsed light device according to another embodiment of the present disclosure. As shown in FIG. 4, the pulsed light device may further include a level setting module 40. A plurality of levels may be set in advance in the level setting module 40, and each level corresponds to a different energy output. By providing the plurality of levels, the pulsed light device can meet the needs of different skin types, target area sizes, hair densities, or other factors.

The control method may further include the following step:
determining a target level in response to a user selecting the target level among the plurality of levels by the level setting module.

In some embodiments, the level setting module may be a physical control panel having physical buttons or knobs. Users may perform a selection operation by pressing or rotating these control elements, to determine the target level.

In some other embodiments, the level setting module may be a touch screen panel which is provided with an interactive interface, and the interactive interface displays different level options. Users may perform a selection operation directly on the interactive interface to select a desired level, to determine the target level.

In still some embodiments, the level setting module may be an application paired with the pulsed light device. Users may pair and communicate with the pulsed light device on which the application is installed through Bluetooth, Wi-Fi, or other wireless communication technologies. In the application, users may browse different level options and select the desired level to determine the target level. This not only realizes remote control but also assists users to make a better choice with additional information provided by the application (such as detailed descriptions of each level, applicable skin types, expected effects, and safety tips). In addition, the application may store the user's usage history and preference settings.

The controlling the light source to flash N times in one flash window may include: controlling the light source to flash N times in one flash window according to the target level, where the plurality of levels are different from one another in one or more of values of N, M, and K, the preset duration, a flash duration of each flash of the light source, and the time interval between adjacent flashes. The energy emitted by the flash of the light source is positively correlated with any one of the values of N, M, and K, the preset duration, the flash duration of each flash of the light source, and the time interval between two adjacent flashes.

In some embodiments, the level setting module is provided with a high level and a low level.

The value of N in the high level is greater than the value of N in the low level. That is, the number of flashes in the high level is greater than the number of flashes in the low level, which can be applied to a wider target area or achieve a better processing effect.

And/or, the value of M in the high level is greater than the value of M in the low level. That is, the number of flashes in the first stage in the high level is greater than the number of flashes in the first stage in the low level, which can preheat the target area more effectively, thereby laying foundation for subsequent treatment.

And/or, the value of K in the high level is greater than the value of K in the low level. That is, the number of flashes in the second stage in the high level is greater than the number of flashes in the second stage in the low level, which can enhance the treatment effect on the target area and make better use of the accumulated thermal effect.

And/or, the value of the preset duration in the high level is greater than the value of the preset duration in the low level. That is, the preset duration in the high level is greater than the preset duration in the low level, which allows the capacitor to be charged more sufficiently, thereby providing higher energy for the next flash.

And/or, the flash duration of each flash of the light source in the high level is longer than that of each flash of the light source in the low level. That is, the flash duration of each flash of the light source in the high level is longer than that of each flash of the light source in the low level. Extending the flash duration can provide a stronger thermal effect, which is suitable for deep treatment.

And/or, the time interval between two adjacent flashes in the high level is greater than the time interval between two adjacent flashes in the low level. That is, the time interval between two adjacent flashes in the high level is greater than the time interval between two adjacent flashes in the low level. Increasing the time interval between two flashes can reduce the risk of overheating, especially when the energy of each flash of the light source is relatively high.

In order to better understand the embodiments of the present disclosure, the change of the voltage of the capacitor in the embodiments of the present disclosure is described below.

Taking a two-stage flashing as an example, the capacitor is fully charged at the initial stage with a maximum voltage of 320 V. When the light source performs M flashes in the first stage, each flash consumes a certain amount of power of the capacitor, causing the voltage to gradually decrease. For example, the capacitor voltage drops to 290 V at the end of the first stage. The charging module is controlled to charge the capacitor for a preset duration, which can fully or partially restore the voltage of the capacitor. Assuming that after charging, the voltage of the capacitor is restored from 300 V to 310 V. Then, the light source is controlled to perform K flashes in the second stage, which further consumes the power of the capacitor, causing the voltage to drop again. For example, the capacitor voltage drops from 310 V to 190 V after the second stage.

In a configuration that M is equal to 2, K is equal to 2, and the preset duration is 0.6 seconds, assuming that the capacitor is fully charged with an initial voltage of 320 V, the light source is controlled to perform the first flash of the first stage, and the voltage of the capacitor drops from 320 V to 305 V. Then the capacitor is recharged to 307 V, the second flash of the first stage is performed, and the voltage of the capacitor further drops from 307 V to 290 V. At this point, the two flashes of the first stage are completed. Subsequently, the charging module is controlled to charge the capacitor for 0.6 seconds, to restore the voltage of the capacitor from 290 V to 310 V. Then, the first flash of the second stage (the third flash) is carried out. The voltage of the capacitor drops from 310 V to 295 V, and then the capacitor is recharged to 297V. The second flash of the second stage (the fourth flash) is performed, and the voltage of the capacitor further drops from 297 V to 190 V. At this point, the two flashes of the second stage are completed.

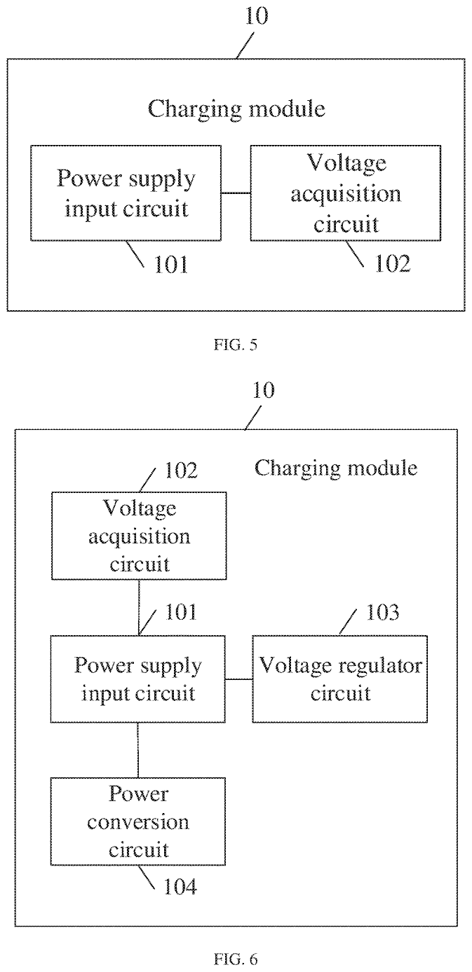

FIG. 5 is a schematic diagram of a charging module according to an embodiment of the present disclosure. As shown in FIG. 5, the charging module 10 may include a power supply input circuit 101 and a voltage acquisition circuit 102. The power supply input circuit 101 and the voltage acquisition circuit 102 are both connected to the capacitor.

The controlling the charging module to charge the capacitor may include the following step:
controlling the power supply input circuit to charge the capacitor.

The power supply input circuit may provide a power for charging the capacitor. The power supply input circuit may be a voltage conversion component or a power supply adapter, etc. The power supply input circuit is connected to an external power source, and outputs a preset power supply to the capacitor after voltage conversion. For example, the power supply input circuit is connected to an external alternating current (AC) or direct current (DC) power supply, and outputs a DC power of 24 V to the capacitor after voltage conversion, so as to provide a DC charging for the capacitor.

In some embodiments, the voltage acquisition circuit may be controlled to acquire a voltage of the capacitor. In a case that the voltage acquired by the voltage acquisition circuit is greater than a preset voltage, the power supply input circuit is controlled to stop charging the capacitor.

The voltage acquisition circuit may monitor a real-time voltage of the capacitor, and feed back the acquired voltage to the pulsed light device. The pulsed light device is set with the preset voltage. When the voltage of the capacitor reaches the preset voltage, it is considered that the capacitor has been sufficiently charged. Therefore, once the voltage acquired by the voltage acquisition circuit is greater than the preset voltage, the power supply input circuit is controlled to stop charging the capacitor to prevent overcharging, thereby ensuring the safety of the capacitor and the pulsed light device.

In some embodiments, the power supply input circuit may be controlled to stop charging the capacitor after the power supply input circuit has charged the capacitor for a preset charging duration.

The pulsed light device may charge the capacitor for the preset charging duration. Even if the voltage of the capacitor does not reach a safety threshold, the charging the capacitor will not continue, which can prevent overcharging due to abnormalities in the device or the capacitor.

FIG. 6 is a schematic diagram of a charging module according to another embodiment of the present disclosure. As shown in FIG. 6, the charging module 10 may further include a voltage regulator circuit 103 and a power conversion circuit 104.

The voltage regulator circuit 103 is connected to the power supply input circuit 101. The voltage regulator circuit 103 is configured to convert a first power supply voltage output by the power supply input circuit 101 into a second power supply voltage and supply the second power supply voltage to the controller. The second power supply voltage may be less than the first power supply voltage. In some embodiments, the first power supply voltage output by the power supply input circuit 101 may be, but is not limited to, 24 V, and the second power supply voltage may be, but is not limited to, 15 V.

The power conversion circuit 104 is connected between the power supply input circuit 101 and the capacitor. The power conversion circuit 104 may convert the first power supply voltage output by the power supply input circuit 101 into a third power supply voltage, and supply the third power supply voltage to the capacitor to charge the capacitor. The third power supply voltage may be greater than the first power supply voltage.

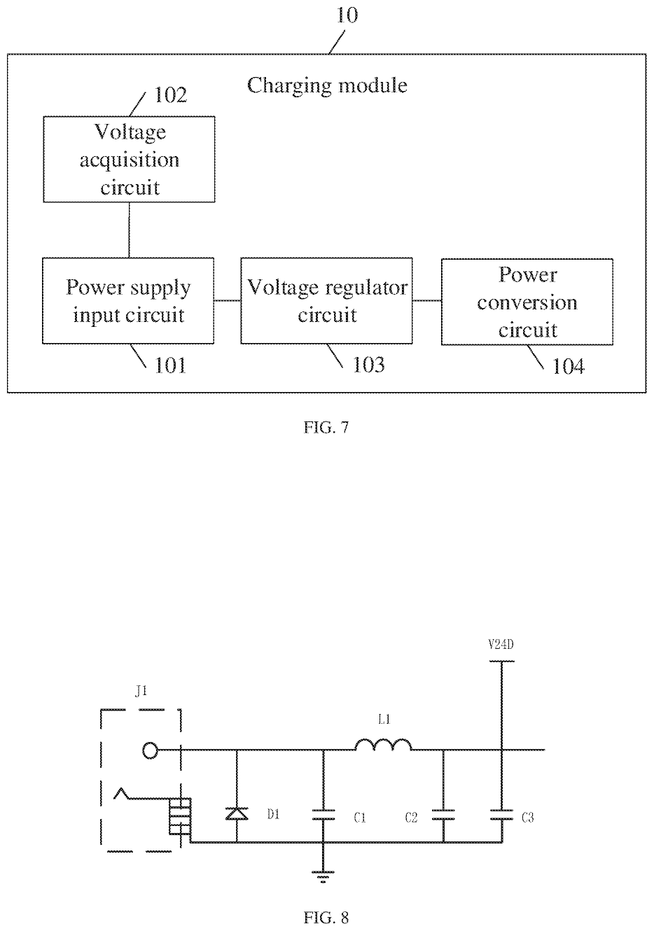

FIG. 7 is a schematic diagram of a charging module according to still another embodiment of the present disclosure. As shown in FIG. 7, the charging module 10 may further include a voltage regulator circuit 103 and a power conversion circuit 104.

The voltage stabilization circuit 103 is connected to the power supply input circuit 101, and the power conversion circuit 104 is connected between the voltage stabilization circuit 103 and the capacitor. The power conversion circuit 104 may convert a second power supply voltage output by the voltage stabilization circuit 103 into a third power supply voltage, and supply the third power supply voltage to the capacitor to charge the capacitor. In some embodiments, the third power supply voltage is adjustable, with a range approximately from 260 V to 320 V.

FIG. 8 is a schematic diagram of a power supply input circuit according to an embodiment of the present disclosure. As shown in FIG. 8, the power supply input circuit may include a power supply access unit J1, a voltage regulator diode DI, a first capacitor C1, a second capacitor C2, a third capacitor C3, and a first inductor L1.

The power supply access unit J1 is configured to receive a power supply from an external power source, and output the first power supply voltage. The power supply access unit J1 is connected to the voltage regulator circuit 103 by the first inductor L1. In some embodiments, the power supply access unit J1 may access an external AC power source or an external DC power source, and output the first power supply voltage to the voltage regulator circuit 103. In some embodiments, the voltage regulator circuit 103 may convert the first power supply voltage output by the power supply access unit JI into the second power supply voltage, and supply the second power supply voltage to the controller. The voltage regulator diode D1 and the first capacitor C1 are arranged in parallel, one end of the parallel connection of the voltage regulator diode D1 and the first capacitor C1 is connected between the power supply access unit JI and the first inductor L1, and the other end is grounded. The second capacitor C2 and the third capacitor C3 are arranged in parallel, one end of the parallel connection of the second capacitor C2 and the third capacitor C3 is connected between the first inductor L1 and the voltage regulator circuit 103, and the other end is grounded. One end of the third capacitor C3 is further connected to the first power supply network V24D.

FIG. 9 is a schematic diagram of a voltage acquisition circuit according to an embodiment of the present disclosure. As shown in FIG. 9, the voltage acquisition circuit 102 includes a first resistor R1, a second resistor R2, a third resistor R3, a fourth resistor R4, a fourth capacitor C4, and an acquisition terminal V400_ADC.

The first resistor R1, the second resistor R2, and the third resistor R3 are successively connected in series. An end of the first resistor R1 is connected to an end of the second resistor R2, the other end of the second resistor R2 is connected to an end of the third resistor R3. The other end of the first resistor R1 is connected to a second power network V400V. The other end of the third resistor R3 is grounded. An end of the fourth resistor R4 is connected between the second resistor R2 and the third resistor R3, and the other end of the fourth resistor R4 may be connected to the acquisition terminal V400_ADC. The acquisition terminal V400_ADC may be connected between the second resistor R2 and the third resistor R3, and configured to acquire a voltage divided between the second resistor R2 and the third resistor R3 to obtain an acquisition voltage. One end of the fourth capacitor C4 is connected to the fourth resistor R4, and the other end of the fourth capacitor C4 is grounded. Thus, the voltage acquisition circuit 102 may acquire the voltage divided between the second resistor R2 and the third resistor R3 to obtain the acquisition voltage, and feed back the acquisition voltage to the controller.

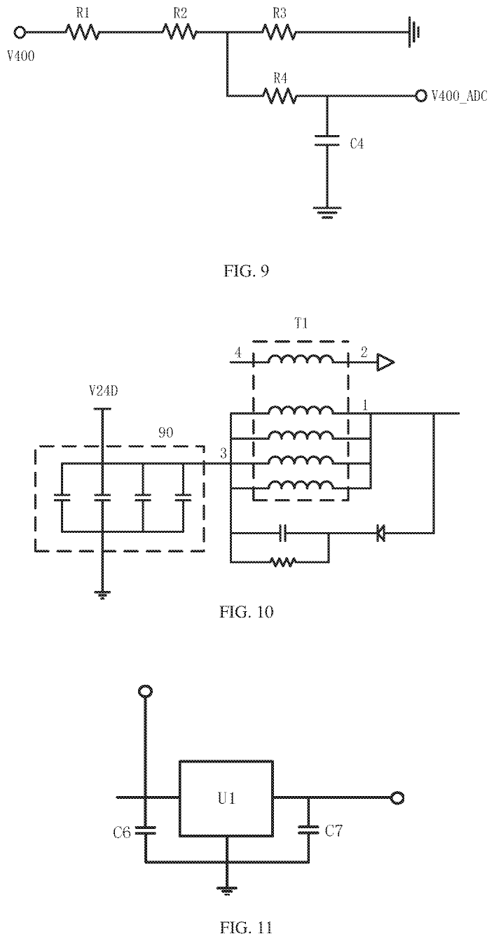

FIG. 10 is a schematic diagram of a power conversion circuit according to an embodiment of the present disclosure. As shown in FIG. 10, the power conversion circuit may include a transformer T1 and an energy storage and filtering sub-circuit 90. A first input terminal 1 of the transformer T1 is connected to a driving pin of a power control unit, and a second input terminal 2 of the transformer T1 is grounded. A first output terminal 3 of the transformer T1 is connected to the energy storage and filtering sub-circuit 90, and a second output terminal 4 of the transformer T1 is connected to the capacitor.

In some embodiments, the energy storage and filtering sub-circuit 90 may be, but is not limited to, a capacitor group, including a plurality of capacitors arranged in parallel. The energy storage and filtering sub-circuit 90 may be configured for energy storage. An end of the plurality of capacitors arranged in parallel may be connected to the first power supply network V24D, and the other end of the plurality of capacitors arranged in parallel may be grounded.

The driving pin of the power control unit may output a driving signal, to allow the transformer to convert the second power supply voltage into the third power supply voltage, and output the third power supply voltage to the capacitor for charging the capacitor. In some embodiments, the power control unit may be a chip having a power control function.

FIG. 11 is a schematic diagram of a voltage regulator circuit according to an embodiment of the present disclosure. As shown in FIG. 11, the voltage regulator circuit may include a voltage regulator unit U1, a sixth capacitor C6, and a seventh capacitor C7. An end of the voltage regulator unit U1 is connected to the power supply input module 10, and the other end of the voltage regulator unit Ul is connected to a power supply pin VCC of the power control unit. An end of the sixth capacitor C6 is connected between the voltage regulator unit U1 and the power supply input module 10, and the other end of the sixth capacitor C6 is grounded. An end of the seventh capacitor C7 is connected between the voltage regulator unit U1 and the power supply pin VCC of the power control unit, and the other end of the seventh capacitor C7 is grounded.

In some embodiments, the voltage regulator unit U1 may be, but is not limited to, a DC-DC converter or a low dropout regulator (LDO).

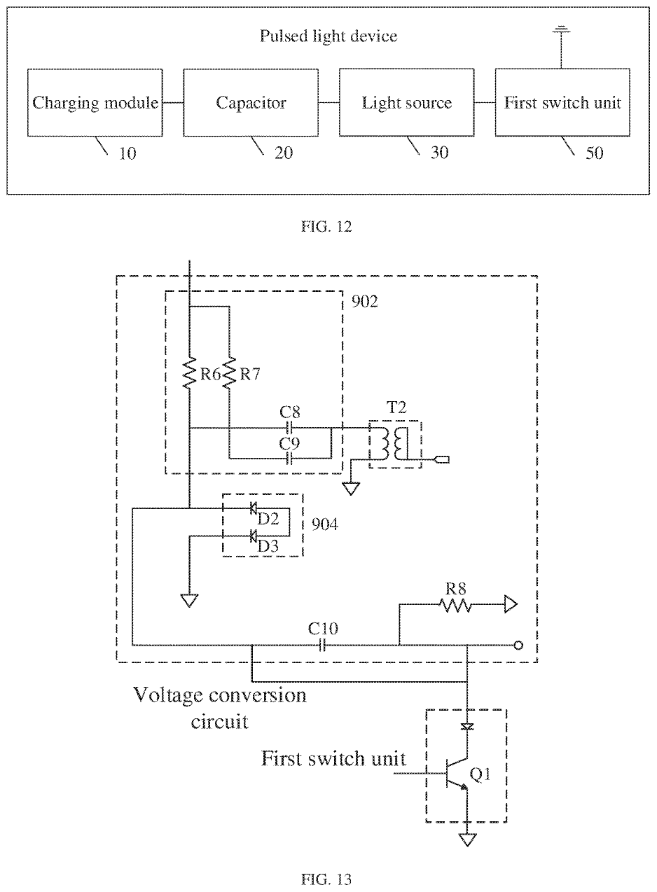

FIG. 12 is a schematic diagram of a pulsed light device according to still another embodiment of the present disclosure. As shown in FIG. 12, the pulsed light device may further include a first switch unit 50. A first terminal of the first switch unit 50 is connected to the light source, and a second terminal of the first switch unit 50 is grounded.

The controlling the light source to flash N times in one flash window may include:
controlling the first terminal and the second terminal of the first switch unit to be turned on, to allow the capacitor to discharge to the light source, so as to allow the light source to flash.

A conduction instruction may be sent to the first switch unit to control conduction between the first terminal and the second terminal of the first switch unit. The capacitor may form a circuit with the ground through the first switch unit, allowing the circuit to be closed. The closed circuit allows the electrical energy stored in the capacitor to flow towards the light source, to provide the necessary energy to the light source to flash.

In some embodiments, the pulsed light device may further include a physical button (not shown), which may detect a user operation to trigger the light source to flash. The controller may generate and output an enable signal to the discharge circuit in response to the user operation, such that the discharge circuit may control the light source to flash in response to the user operation.

In some embodiments, the pulsed light device may further include a voltage conversion circuit. The capacitor may be connected to the light source by the voltage conversion circuit, and the capacitor may also be connected to the first switch unit by the voltage conversion circuit. The capacitor may be configured to store and release electric energy, and the electrical energy released by the capacitor may be provided to the light source as the working electrical energy of the light source. The first switch unit may be configured to conduct the voltage conversion circuit in response to receiving the enable signal. In some embodiments, the first switch unit may be controlled by the enable signal to switch between different states, such as an on state or an off state, so as to control the voltage conversion circuit to be on or off.

FIG. 13 is a schematic diagram of a voltage conversion circuit according to an embodiment of the present disclosure. As shown in FIG. 13, the voltage conversion circuit may include a storage unit 902 and a transformer T2.

The storage unit 902 is connected to the capacitor, and may be configured to obtain a first operating voltage from the capacitor to store electric energy. The transformer T2 is connected between the storage unit 902 and the light source. The transformer T2 may be configured to obtain the first operating voltage from the storage unit 902, convert the first operating voltage to a second operating voltage, and output the second operating voltage to the light source, to allow the light source to flash. In some embodiments, the storage unit 902 obtains the electrical energy from the capacitor and stores it temporarily. When the storage unit 902 releases the electrical energy to the transformer T2, the storage unit 902 outputs the electrical energy at the first operating voltage.

The storage unit 902 may include an eighth capacitor C8, a ninth capacitor C9, a sixth resistor R6, and a seventh resistor R7.

The sixth resistor R6 and the eighth capacitor C8 are connected between the capacitor and the input terminal of the transformer T2, the seventh resistor R7 and the ninth capacitor C9 are connected between the capacitor and the input terminal of the transformer T2, and the circuit formed by the seventh resistor R7 in series with the ninth capacitor C9 is connected in parallel with the circuit formed by the sixth resistor R6 in series with the eighth capacitor C8. The eighth capacitor C8 and the ninth capacitor C9 are configured to obtain the first operating voltage output by the capacitor, to store the electric energy. In some embodiments, one end of the sixth resistor R6 and one end of the seventh resistor R7 may be connected to the first power supply network V400V, and the capacitor is also connected to the first power supply network V400V, such that the one end of the sixth resistor R6 and the one end of the seventh resistor R7 may be connected to the capacitor by the first power supply network V400V.

The transformer T2 includes an input terminal, an output terminal, and a ground terminal. The output terminal of the transformer T2 is connected to the light source, and the ground terminal of the transformer T2 is grounded.

The first switch unit may include, but is not limited to, an insulate-gate bipolar transistor (IGBT).

In a configuration that the first switch unit is an IGBT, the IGBT may include a control terminal, a first terminal, and a second terminal. The following description uses the first switch unit being an IGBT Q1 as an example. The control terminal of the IGBT Q1 is connected to the driving circuit, and may receive the enable signal through the driving circuit. The first terminal of the IGBT Q1 is connected to the voltage conversion circuit, and the second terminal of the IGBT Q1 is grounded. When the control terminal of the IGBT Q1 receives the enable signal, the first terminal and the second terminal of the IGBT Q1 are conducted.

The IGBT Q1 may be configured as: when the control terminal of the IGBT Q1 receives the enable signal, the IGBT is turned on, so that the eighth capacitor C8 and the ninth capacitor C9 output the stored electric energy to the input terminal of the transformer T2, the transformer T2 converts the first operating voltage to the second operating voltage and outputs the second operating voltage to the light source through the output terminal, allowing the light source to flash.

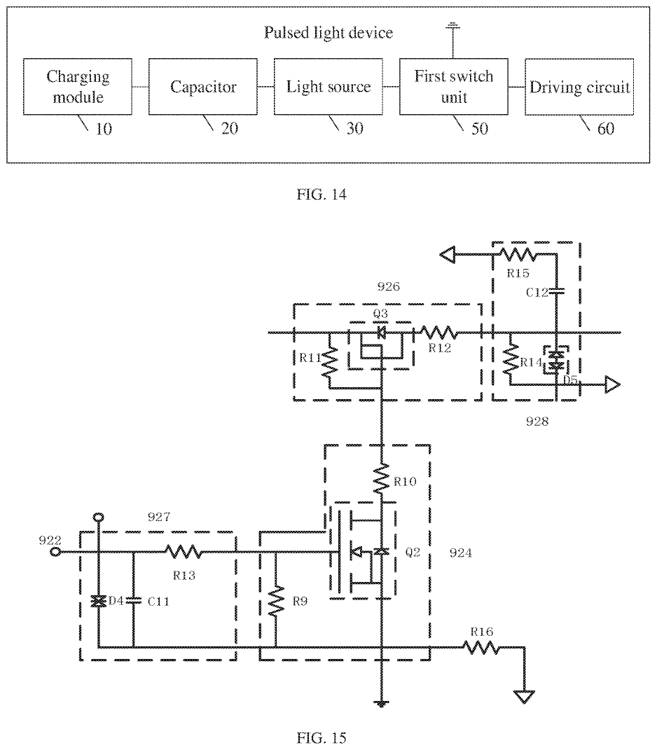

FIG. 14 is a schematic diagram of a pulse light device according to still another embodiment of the present disclosure. The pulsed light device may further include a driving circuit 60 connected to the first switch unit 50.

The driving circuit may receive the enable signal output by the controller, and control the first switch unit to be turned on or off.

FIG. 15 is a schematic diagram of a driving circuit according to an embodiment of the present disclosure. As shown in FIG. 15, the driving circuit may include a signal receiving terminal 922, a third switch unit 924, and a fourth switch unit 926.

The signal receiving terminal 922 may receive the enable signal. The third switch unit 924 is connected between the signal receiving terminal 922 and the fourth switch unit 926. The fourth switch unit 926 is connected between the third switch unit 924 and the first switch unit. When the signal receiving terminal 922 receives the enable signal, the third switch unit 924 is turned on, and the fourth switch unit 926 is turned on, so as to control the first switch unit to be turned on.

The third switch unit 924 may include a second switch Q2, a ninth resistor R9, and a tenth resistor R10. A controlled terminal of the second switch Q2 is connected to the signal receiving terminal 922, a first terminal of the second switch Q2 is grounded, and a second terminal of the second switch Q2 is connected to the fourth switch unit 926 by the tenth resistor R10. The ninth resistor R9 is connected between the controlled terminal and the first terminal of the second switch Q2. When the signal receiving terminal 922 receives the enable signal, the first terminal and the second terminal of the second switch Q2 are conducted. In some embodiments, the second switch Q2 may be, but is not limited to, a metal-oxide-semiconductor (MOS) field-effect transistor, where the controlled terminal of the second switch Q2 may be a gate, the first terminal of the second switch Q2 may be a source, and the second terminal of the second switch Q2 may be a drain.

The fourth switch unit 926 may include a third switch Q3, an eleventh resistor R11, and a twelfth resistor R12. A controlled terminal of the third switch Q3 is connected to the second terminal of the second switch Q2 by the tenth resistor R10, a first terminal of the third switch Q3 is connected to the controlled terminal by the eleventh resistor R11, and a second terminal of the third switch Q3 is connected to the first switch unit by the twelfth resistor R12. In some embodiments, the second terminal of the third switch Q3 is connected to the control terminal of the IGBT Q1 by the twelfth resistor R12. When the first terminal and the second terminal of the second switch Q2 are conducted, the first terminal and the second terminal of the third switch Q3 are conducted. In some embodiments, the third switch Q3 may be, but is not limited to, a MOS transistor, where the controlled terminal of the third switch Q3 may be a gate, the first terminal of the third switch Q3 may be a source, and the second terminal of the third switch Q3 may be a drain.

As shown in FIG. 15, the driving circuit may further include a first filtering unit 927 and a first protection unit 928.

The first filtering unit 927 is connected between the signal receiving terminal 922 and the third switch unit 924. The first filtering unit 927 may be configured to filter the signal received by the signal receiving terminal 922, such as the enable signal. The first protection unit 928 is connected between the fourth switch unit 926 and the first switch unit. The first protection unit 928 may be configured to protect the first switch unit, for example, to prevent the first switch unit from being impacted by over-current or over-voltage signals.

The first filtering unit 927 may include a fourth diode D4, an eleventh capacitor C11, and a thirteenth resistor R13. The thirteenth resistor R13 is connected between the signal receiving terminal 922 and the controlled terminal of the first switch Q2. One end of the fourth diode D4 is connected between the signal receiving terminal 922 and the thirteenth resistor R13, and the other end of the fourth diode D4 is grounded. The eleventh capacitor C11 is connected in parallel with the fourth diode D4. One end of the eleventh capacitor C11 is connected between the signal receiving terminal 922 and the thirteenth resistor R13, and the other end of the eleventh capacitor C11 is grounded. In some embodiments, the other end of the eleventh capacitor C11 and the other end of the fourth diode D4 may be grounded by a sixteenth resistor R16.

The first protection unit 928 includes a rectifier D5, a fourteenth resistor R14, a twelfth capacitor C12, and a fifteenth resistor R15. One end of the rectifier D5 is connected between the twelfth resistor R12 and the first switch unit, and is also grounded by the twelfth capacitor C12 and the fifteenth resistor R15; the other end of the rectifier D5 is grounded. In some embodiments, the one end of the rectifier D5 is connected between the twelfth resistor R12 and the control terminal of the IGBT Q1. The fourteenth resistor R14 is connected in parallel with the rectifier D5. One end of the fourteenth resistor R14 is connected between the twelfth resistor R12 and the first switch unit, and the other end of the fourteenth resistor R14 is grounded. In some embodiments, the one end of the fourteenth resistor R14 is connected between the twelfth resistor R12 and the control terminal of the IGBT Q1.

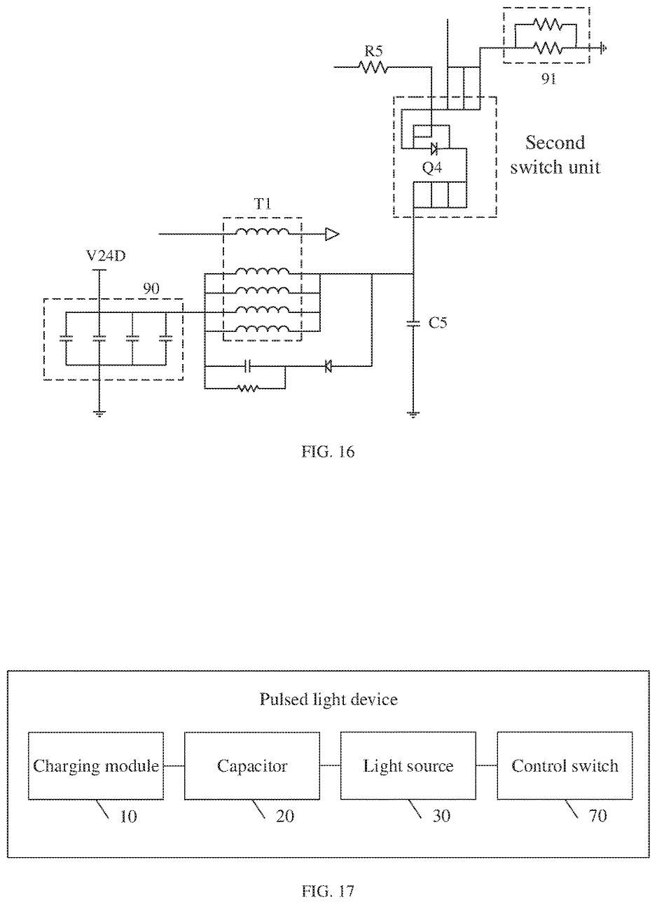

FIG. 16 is a schematic diagram of a charging module according to still another embodiment of the present disclosure. As shown in FIG. 16, the charging module may further include a second switch unit, and the end of the capacitor connected to the light source is grounded. The second switch unit may be connected between the power control unit and the transformer T1.

The second switch unit may include, but is not limited to, a MOS transistor.

In a configuration that the second switch unit is a MOS transistor, the MOS transistor may include a control terminal, a first terminal, and a second terminal. The following uses the second switch unit being a MOS transistor Q4 as an example.

The control terminal of the MOS transistor Q4 is connected to the driving pin of the power control unit by the fifth resistor R5. The first terminal of the MOS transistor Q4 is connected to the first input terminal of the transformer T1, and the first terminal of the MOS transistor Q4 is also grounded by a fifth capacitor C5. The second terminal of the MOS transistor Q4 is grounded by a resistor group 91. In some embodiments, the resistor group 91 may include a plurality of resistors arranged in parallel, for example, two resistors arranged in parallel.

Based on the foregoing structure, the control method in the present disclosure may further include: receiving a flashing signal.

According to the flashing signal, a charging signal may be sent to the charging module, and a discharging signal may be sent to the first switch unit, to control the first switch unit and the second switch unit to be on or off in a preset manner. The first switch unit has an on-off state opposite to that of the second switch unit to realize the following: controlling the light source to flash N times in one flash window, where N is an integer greater than or equal to 3; controlling the capacitor to discharge to the light source during each flash of the light source; and controlling the charging module to charge the capacitor during a time interval between two adjacent flashes.

The on-off state of the first switch unit is opposite to that of the second switch unit. That is, when the first switch unit is on, the second switch unit is controlled to be off. When the capacitor discharges to the light source to allow the light source to flash, the connection between the charging module and the capacitor is cut off to prevent the capacitor from being charged simultaneously. After the flashing of the light source is completed, the first switch unit is controlled to be off, and the second switch unit is controlled to be on. At this time, the capacitor starts to be charged to store energy for the next flash of the light source.

The on-off states of the first switch unit and the second switch unit may be controlled according to the flashing signal, to manage the charging and discharging of the pulse light device.

FIG. 17 is a schematic diagram of a pulsed light device according to still another embodiment of the present disclosure. As show in FIG. 17, the pulsed light device may further include a control switch 70 connected to the light source.

The controlling the capacitor to discharge to the light source may include: controlling the control switch to be on, to allow the light source to flash.

In continuous flashing of the light source, maintaining an appropriate time interval can prevent the light source from overheating. The overheating of the light source may not only damage the device but may also cause unnecessary harm to the user's skin. To ensure that the device has enough time to cool down and avoid overheating issues, the flashing of the light source may be controlled by controlling the on and off state of the switch. For example, in the flash window, the working state of the light source is controlled by controlling the on and off state of the control switch, which can protect the device from damage and ensure the safety and comfort of the user.

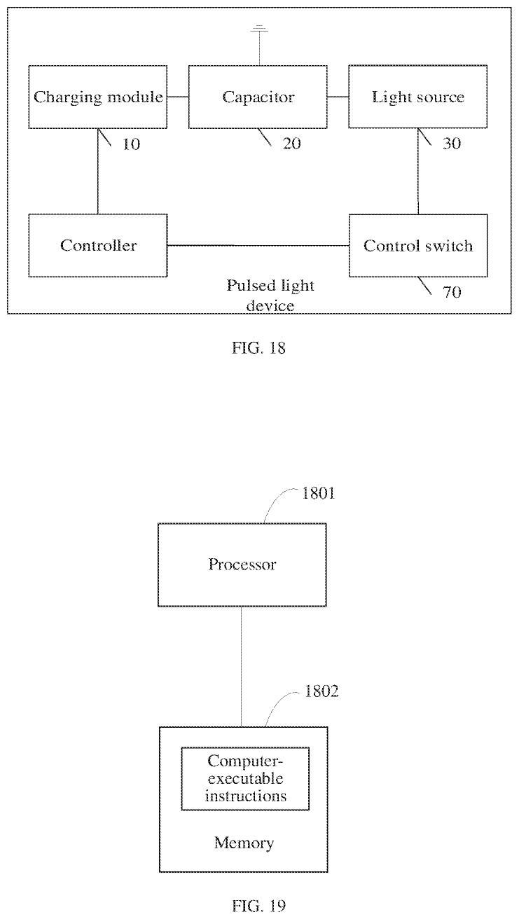

FIG. 18 is a schematic diagram of a pulsed light device according to still another embodiment of the present disclosure. As shown in FIG. 18, the pulsed light device may include a controller, a charging module 10, a capacitor 20, a light source 30, and a control switch 70.

When controlling the charging module to charge the capacitor, the controller may send a charging instruction to the charging module 10, to allow the charging module 10, the capacitor 20, and the ground to form a circuit, thereby realizing the charging of the capacitor 20. The charging module 10 may charge the capacitor 20 by the power supply input circuit, and monitor the voltage of the capacitor 20 by the voltage acquisition circuit. When the voltage of the capacitor 20 reaches a preset voltage or the charging duration reaches a preset duration (for example, 0.6 seconds), the controller may send a stop charging instruction to the charging module 10.

When controlling the light source to flash, the controller may send a discharging instruction to the control switch 70, and the control switch 70 is turned on in response to receiving the discharging instruction, to allow the light source 30 to flash.

By way of the foregoing method and structure, it is achieved that the capacitor discharges to the light source to allow the light source to flash, and the charging module charges the capacitor during the time interval between two adjacent flashes.

In some embodiments, the controller sends a pulse width modulation (PWM) signal to the charging module and the control switch, to control the charging module to charge the capacitor and control the light source to flash.

The charging module may charge the capacitor according to the PWM signal. For example, the controller sends the PWM signal to the charging module, when the PWM is at a high level, the charging module, the capacitor, and the ground form a circuit, thereby realizing the charging of the capacitor; and when the PWM signal returns to a low level, the charging module stops charging the capacitor. The charging duration of the capacitor may depend on the width (the high-level duration) of the PWM signal.

The control switch may control the light source to flash according to the PWM signal. For example, the controller sends a PWM signal to the control switch, when the PWM is at a high level, the control switch is turned on, and the light source starts to flash; and when the PWM signal returns to a low level, the light source is controlled to stop flashing. The duration of each flash may depend on the width (the high-level duration) of the PWM signal.

In some other embodiments, a timer is provided in the controller, to ensure that all actions are performed according to a preset sequence.

When controlling the capacitor to discharge to the light source, the controller may send a working signal to the control switch according to a preset time of the timer, to allow the control switch to be on, thereby allowing the light source to flash. After the working signal ends, the control switch is turned off, and the light source stops flashing. The duration of each flash may depend on the duration of the working signal.

When controlling the charging module to charge the capacitor, the controller may send a working signal to the charging module according to a preset time of the timer, to allow the charging module, the capacitor, and the ground to form a circuit, thereby realizing the charging of the capacitor. After the working signal ends, the charging module stops charging. The charging duration of the capacitor may depend on the duration of the working signal.

It should be understood that the same or corresponding information in the foregoing different embodiments may be cross-referenced.

According to some embodiments, the present disclosure further provides a control apparatus for a pulsed light device. The pulsed light device may include a charging module, a capacitor, and a light source. The capacitor is connected between the charging module and the light source, the charging module may charge the capacitor, and the capacitor may supply power to the light source to allow the light source to flash. The control apparatus may include a control module, which is configured to:
control the light source to flash N times in one flash window, where N is an integer greater than or equal to 3;
control the capacitor to discharge the light source during each flash of the light source; and
control the charging module to charge the capacitor during a time interval between two adjacent flashes.

In some embodiments, the control module is further configured to control the light source to flash M times, where M is an integer greater than or equal to 1; and/or, control the charging module to charge the capacitor for a preset duration, where the preset duration is greater than or equal to 0.4 seconds and less than or equal to 0.95 seconds; and/or, control the light source to flash K times, where K is an integer greater than or equal to 1, and N is greater than or equal to the sum of M and K.

In some embodiments, the pulsed light device further includes a level setting module which is provided with a plurality of levels. The control apparatus may further include a response module, which is configured to:
determine a target level in response to a user selecting the target level among the plurality of levels by the level setting module.

The control module is further configured to control the light source to flash N times in one flash window according to the target level.

The plurality of levels are different from one another in one or more of values of N, M, and K, the preset duration, a flash duration of each flash of the light source, and the time interval between adjacent flashes.

In some embodiments, the charging module includes a power supply input circuit and a voltage acquisition circuit. The power supply input circuit and the voltage acquisition circuit are both connected to the capacitor. The charging module is controlled to charge the capacitor. The control module is further configured to control the power supply input circuit to charge the capacitor; and/or, control the voltage acquisition circuit to acquire the voltage of the capacitor; and/or, control the power supply input circuit to stop charging the capacitor, in a case that the voltage of the capacitor is greater than a preset voltage or after the power supply input circuit has charged the capacitor for a preset duration.

In some other embodiments, the pulsed light device further includes a first switch unit, a first terminal of the first switch unit is connected to the light source, and a second terminal of the first switch unit is grounded. The first terminal and the third terminal of the first switch unit are controlled to be conducted, so that the capacitor discharges to the light source, to allow the light source to flash.

In some embodiments, the charging module may further include a second switch unit, and the end of the capacitor connected to the light source is grounded.

The control apparatus may also include a receiving module, which may be configured to:
receive a flashing signal.

The control module may send, according to the flashing signal, a charging signal to the charging module and send a discharging signal to the first switch unit, to control the first switch unit and the second switch unit to be turned on or off in a preset manner. The first switch unit has an on-off state opposite to that of the second switch units to realize the following: controlling the light source to flash N times in one flash window, where N is an integer greater than or equal to 3; controlling the capacitor to discharge to the light source during each flash of the light source; and controlling the charging module to charge the capacitor during a time interval between two adjacent flashes.

In some embodiments, the pulsed light device may further include a control switch connected to the light source. The control module may control the control switch to be on, to allow the light source to flash.

Those skilled in the art may clearly understand that, for convenience and brevity of description, the specific working process of the control apparatus, the control module, the response module, and the receiving module described above may refer to the corresponding process in the foregoing method embodiments and content of the first aspect in the summary section, which is not detailed herein.

In the embodiments of the present disclosure, the coupling between the units may be electrical, mechanical, or in other forms.

In addition, the functional units in the embodiments of the present disclosure may be integrated into one processing unit, or each unit may exist alone physically, or two or more units may be integrated into one unit. The integrated unit may be implemented in the form of hardware or in the form of a software functional unit.

The pulsed light device provided in the embodiments of the present disclosure includes a controller, a charging module, a capacitor, and a light source. The capacitor is connected between the charging module and the light source, the charging module is configured to charge the capacitor, and the capacitor is configured to supply power to the light source, to allow the light source to flash.

Those skilled in the art may clearly understand that, for convenience and brevity of description, the specific working process of the controller, the charging module, the capacitor, and the light source described above may refer to the corresponding process in the foregoing method embodiments, which is not detailed herein.

In the embodiments of the present disclosure, the coupling between the units may be electrical, mechanical, or in other forms.

In addition, the functional units in the embodiments of the present disclosure may be integrated into one processing unit, or each unit may exist alone physically, or two or more units may be integrated into one unit. The integrated unit may be implemented in the form of hardware or in the form of a software functional unit.

FIG. 19 is a schematic diagram of a pulse light device according to still another embodiment of the present disclosure. As shown in FIG. 19, the service terminal may include a processor 1801 and a memory 1802. The memory 1802 may store one or more computer-executable instructions. The one or more computer-executable instructions are configured for performing the method described in the foregoing method embodiments. The memory 1802 may exist separately, or may be integrated with the processor 1801.

The processor 1801 may include one or more processing cores. The processor 1801 may use various interfaces and lines to connect various parts in the entire service terminal, and may execute various functions of the service terminal and process data by running or executing instructions, programs, code sets, or instruction sets stored in the memory 1802, and call data stored in the memory 1802. In some embodiments, the processor 1801 may be implemented by at least one hardware form of digital signal processing (DSP), field programmable gate array (FPGA), and programmable logic array (PLA). The processor 1801 may be integrated with a central processing (CPU), a graphics processing unit (GPU), a modem, or the like, or a combination thereof. The CPU mainly processes an operating system, a user interface, an application program, and the like. The GPU is configured to render and draw display content. The modem is configured to process wireless communication. It can be understood that the modem may be implemented by using a communication chip alone rather than be integrated into the processor 1801.

The memory 1802 may include a random-access memory (RAM) or a read-only memory (ROM). The memory 1802 may be configured to store instructions, programs, codes, code sets, or sets of instruction sets. The memory 1802 may include a program storage area and a data storage area, where the program storage area may store an instruction for implementing an operating system, an instruction for implementing at least one function, an instruction for implementing the foregoing method embodiments, and the like. The data storage area may further store data created by the service terminal in use, and the like.

When the computer-executable instructions stored in the memory 1802 are executed, the processor 1801 may be configured to perform various operations performed by the service terminal in the foregoing method embodiments. The specific implementation of these operations may refer to the foregoing embodiments, which is not detailed herein.

An embodiment of the present disclosure further discloses a computer-readable storage medium, storing computer-executable instruction codes. The computer-executable instruction codes when being executed by a processor are configured to perform the operations carried out by the service terminal in the foregoing method embodiments. The specific implementation of these operations may refer to the foregoing method embodiments, which is not detailed herein.

The computer-readable storage medium may be an electronic storage device such as flash memory, an erasable programmable read-only memory (EPROM), a hard disk, or a read-only memory (ROM). In some embodiments, the computer-readable storage medium may include a non-transitory computer-readable storage medium. The computer-readable storage medium has a storage space for the computer-executable instruction codes that execute the operations in the any of the foregoing method embodiments. These computer-executable instruction codes may be read from one or more computer-executable instructions products or written into one or more computer-executable instruction products. The computer-executable instruction codes may be, for example, appropriately compressed.

It should be noted that the above embodiments are only used to illustrate the technical solutions of the present disclosure, but not to limit them. Although a detailed description of the present disclosure has been given with reference to the foregoing embodiments, those skilled in the art should understand that they can still modify the technical solutions disclosed in the foregoing embodiments, or equivalently substitute some of the technical features. These modifications or substitutions do not cause the essence of the corresponding technical solutions to deviate from the spirit and scope of the technical solutions of the embodiments of the present disclosure.

What is claimed is:

1. A control method for a pulsed light device, wherein the pulsed light device is a hair removal device or a skin rejuvenation device, and the pulsed light device comprises a charging module, a capacitor, and a light source, the capacitor is connected between the charging module and the light source, the charging module is configured to charge the capacitor, and the capacitor is configured to supply power to the light source to allow the light source to flash; and
the control method comprises:
controlling the light source to flash N times in one flash window, N being an integer greater than or equal to 3; wherein a flash duration of each flash of the light source is less than or equal to 10 milliseconds, delivering energy to a skin through superposition and accumulation;
controlling the capacitor to discharge to the light source during each flash of the light source; and
controlling the charging module to charge the capacitor during a time interval between two adjacent flashes;
wherein the controlling the light source to flash N times in one flash window comprises:
controlling the light source to flash M times, M being an integer greater than or equal to 1;
controlling the charging module to charge the capacitor for a preset duration, the preset duration being greater than or equal to 0.4 seconds and less than or equal to 0.95 seconds; and
controlling the light source to flash K times, K being an integer greater than or equal to 1, and N being greater than or equal to the sum of M and K.

2. The control method according to claim 1,
wherein in a configuration that M is greater than 1 and K is greater than 1, a time interval between adjacent flashes in the M flashes and a time interval between adjacent flashes in the K flashes are both less than or equal to the preset duration.

3. (canceled)

4. (canceled)

5. The control method according to claim 1, wherein
in a configuration that M is greater than 1, a time interval between adjacent flashes in the M flashes is greater than or equal to 0.06 seconds and less than or equal to 0.4 seconds; and/or,
the preset duration is greater than or equal to 0.5 seconds and less than or equal to 0.7 seconds; and/or
in a configuration that K is greater than 1, a time interval between adjacent flashes in the K flashes is greater than or equal to 0.06 seconds and less than or equal to 0.4 seconds.

6. The control method according to claim 5, wherein
a time interval between adjacent flashes in the M flashes is greater than or equal to 0.06 seconds and less than or equal to 0.4 seconds comprises:
the time interval between adjacent flashes in the M flashes is greater than or equal to 0.1 seconds and less than or equal to 0.3 seconds; and/or,
a time interval between adjacent flashes in the K flashes is greater than or equal to 0.06 seconds and less than or equal to 0.4 seconds comprises:
the time interval between adjacent flashes in the K flashes is greater than or equal to 0.1 seconds and less than or equal to 0.3 seconds.

7. The control method according to claim 1, wherein
the flash duration of each flash of the light source is greater than or equal to 0.3 milliseconds.

8. The control method according to claim 7, wherein the flash duration of each flash of the light source is greater than or equal to 0.5 milliseconds and less than or equal to 4 milliseconds.

9. The control method according to claim 1, wherein
in a configuration that M is equal to 2, a flash duration of a first flash in the M flashes is equal to or less than a flash duration of a second flash in the M flashes; and/or,
in a configuration that K is equal to 2, a flash duration of a first flash in the K flashes is equal to or less than a flash duration of a second flash in the K flashes.

10. The control method according to claim 1, wherein
in a configuration that K is equal to 2, a flash duration of a first flash in the K flashes is less than a flash duration of a second flash in the K flashes, the flash duration of the first flash in the K flashes is greater than or equal to 0.3 milliseconds and less than or equal to 0.7 milliseconds, and the flash duration of the second flash in the K flashes is greater than or equal to 1.2 milliseconds and less than or equal to 3 milliseconds.

11. The control method according to claim 6, wherein the pulsed light device further comprises a level setting module provided with a plurality of levels;
the control method further comprises:
determining a target level in response to a user selecting the target level among the plurality of levels by the level setting module; and
the controlling the light source to flash N times in one flash window comprises:
controlling the light source to flash N times in one flash window according to the target level;
wherein the plurality of levels are different from one another in one or more of values of N, M, and K, the preset duration, a flash duration of each flash of the light source, and the time interval between adjacent flashes.

12. The control method according to claim 1, wherein the charging module comprises a power supply input circuit and a voltage acquisition circuit, the power supply input circuit and the voltage acquisition circuit are both connected to the capacitor; and
the controlling the charging module to charge the capacitor comprises:
controlling the power supply input circuit to charge the capacitor;
controlling the voltage acquisition circuit to acquire a voltage of the capacitor; and
controlling the power supply input circuit to stop charging the capacitor in a case that the voltage of the capacitor is greater than a preset voltage, or controlling the power supply input circuit to stop charging the capacitor after the power supply input circuit has charged the capacitor for a preset duration.

13. The control method according to claim 1, wherein the pulsed light device further comprises a first switch unit, a first terminal of the first switch unit is connected to the light source, and a second terminal of the first switch unit is grounded; and
the controlling the light source to flash N times in one flash window comprises:
controlling the first terminal and the second terminal of the first switch unit to be conducted, to allow the capacitor to discharge to the light source, so as to allow the light source to flash.

14. The control method according to claim 13, wherein the charging module further comprises a second switch unit, and an end of the capacitor connected to the light source is grounded; and
the control method further comprises:
receiving a flashing signal;
sending, according to the flashing signal, a charging signal to the charging module and a discharging signal to the first switch unit, to respectively control the first switch unit and the second switch unit to be turned on or off in a preset manner, wherein the first switch unit has an on-off state opposite to that of the second switch unit to realize the following:
controlling the light source to flash N times in one flash window, N being an integer greater than or equal to 3;
controlling the capacitor to discharge to the light source during each flash of the light source; and
controlling the charging module to charge the capacitor during a time interval between two adjacent flashes.

15. The control method according to claim 1, wherein the pulsed light device further comprises a control switch connected to the light source; and
the controlling the capacitor to discharge to the light source comprises:
controlling the control switch to be turned on, to allow the light source to flash.

16. A control apparatus for a pulsed light device, wherein the pulsed light device is a hair removal device or a skin rejuvenation device, the pulsed light device comprises a charging module, a capacitor, and a light source, the capacitor is connected between the charging module and the light source, the charging module is configured to charge the capacitor, the capacitor is configured to supply power to the light source to allow the light source to flash; and
the control apparatus comprises a control module configured to:
control the light source to flash N times in one flash window, N being an integer greater than or equal to 3; wherein a flash duration of each flash of the light source is less than or equal to 10 milliseconds, and delivering energy to a skin through superposition and accumulation;
control the capacitor to discharge to the light source during each flash of the light source; and
control the charging module to charge the capacitor during a time interval between two adjacent flashes;
the control module is further configured to:
control the light source to flash M times, M being an integer greater than or equal to 1;
control the charging module to charge the capacitor for a preset duration, the preset duration being greater than or equal to 0.4 seconds and less than or equal to 0.95 seconds; and
control the light source to flash K times, K being an integer greater than or equal to 1, and N being greater than or equal to the sum of M and K.

17. A pulsed light device, wherein the pulsed light device is a hair removal device or a skin rejuvenation device, the pulsed light device comprises a controller, a charging module, a capacitor, and a light source, wherein the capacitor is connected between the charging module and the light source, the charging module is configured to charge the capacitor, the capacitor is configured to supply power to the light source to allow the light source to flash, and the controller is configured to:
control the light source to flash N times in one flash window, N being an integer greater than or equal to 3; wherein a flash duration of each flash of the light source is less than or equal to 1 milliseconds, and delivering energy to a skin through superposition and accumulation;
control the capacitor to discharge to the light source during each flash of the light source; and
control the charging module to charge the capacitor during a time interval between two adjacent flashes;
the controller is further configured to:
control the light source to flash M times, M being an integer greater than or equal to 1;
control the charging module to charge the capacitor for a preset duration, the preset duration being greater than or equal to 0.4 seconds and less than or equal to 0.95 seconds; and
control the light source to flash K times, K being an integer greater than or equal to 1, and N being greater than or equal to the sum of M and K.

18. A pulsed light device, comprising a processor and a memory, the processor calling computer-executable instructions stored in the memory to implement the control method according to claim 1.

19. A computer-readable storage medium, storing computer-executable instructions, the computer-executable instructions implementing the control method according to claim 1 when being executed by a processor.
